(19) United States
(12) Patent Application Publication
(10) Pub. No.: US 2007/0122415 A1
(43) Pub. Date: May 31, 2007

(54) Compound and method for suppressing retroviral replication

(75) Inventors: Phalguni Gupta, Pittsburgh, PA (US); Ashwin Tumne, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh of the Commonwealth System of Higher Education, Pittsburgh, PA (US)

(21) Appl. No.: 11/607,256

(22) Filed: Nov. 29, 2006

Related U.S. Application Data
(60) Provisional application No. 60/740,922, filed on Nov. 29, 2005.

(51) Int. Cl. A61K 39/42; C12Q 1/70; C07K 16/10

(52) U.S. Cl. 424/160.1; 435/5; 530/388.3

(57) ABSTRACT

In one aspect, the invention provides an antiretroviral peptide that suppresses replication of a retrovirus and the use thereof to inhibit retroviral replication within cells infected with a retrovirus. The method can be used in vivo to treat retroviral infection in human or veterinary subjects, and the inventive antiretroviral peptide can be formulated in pharmaceutical compositions to facilitate such method. In another aspect, the invention provides a method for extracting peptides localized to cell exosomes.

### CROSS-REFERENCE TO RELATED APPLICATIONS

This patent application claims the benefit of U.S. Provisional Patent Application No. 60/740,922, filed Nov. 29, 2005, the contents of which are incorporated herein.

### BACKGROUND OF THE INVENTION

Retroviruses are enveloped viruses possessing an RNA genome, and replicate via a DNA intermediate. Retroviruses rely on the enzyme reverse transcriptase to perform the reverse transcription of its genome from RNA into DNA, which can then be integrated into the host's genome with an integrase enzyme. Retroviruses are responsible for numerous human and animal infections that are typically very difficult to treat and are incurable. For instance, the human immunodeficiency virus (i.e., HIV-1 and HIV-2), the virus that causes acquired immune deficiency syndrome (AIDS), is a retrovirus that affects the body's immune system and infects millions of people worldwide and has killed more than 25 million people since its identification in 1981.

Decades of research have led to the production of treatments of retroviral infection, including HIV. While such agents are able to suppress HIV replication, they present the risk of side effects for some patients, and some strains of HIV can mutate to develop resistance to such agents. Thus, there yet remains a need for additional agents that can repress replication of retroviruses, including HIV.

In the realm of research into anti-retroviral agents, it is known that host mechanisms are active, to some degree, in suppressing the replication of retrovirus in infected cells. For example, T lymphocytes (CD8+, CD4+) and B lymphocytes play important roles in retroviral suppression. However, despite decades of intensive investigation, the effector compound responsible for such suppression has not been conclusively identified. One hurdle in the identification and isolation of such factors is the lack of an efficient method of isolating such factors from cellular fractions. Methods within the current state of the art result in extractions from cell fractions that contain too many impurities, such that correlating biological activity to a particular factor (typically proteinaceous) is not feasible without substantial additional effort at isolation. Accordingly, there is a need for an improved method of isolating proteins from cellular fractions.

### BRIEF SUMMARY OF THE INVENTION

In one aspect, the invention provides an antiretroviral peptide that suppresses replication of a retrovirus and the use thereof to inhibit retroviral replication within cells infected with a retrovirus. The method can be used in vivo to treat retroviral infection in human or veterinary subjects, and the inventive antiretroviral peptide can be formulated in pharmaceutical compositions to facilitate such method.

In another aspect, the invention provides a method for extracting peptides localized to cell exosomes.

These aspects and other inventive features will be apparent from the accompanying drawings and the following detailed description.

### BRIEF DESCRIPTION OF THE SEVERAL VIEWS OF THE DRAWINGS

FIG. 1A is a graph showing the % suppression of extracellular HIV-1 p24 versus CD8+ effector: HIV-1 infected CD4+ cell ratio.

FIG. 1B is a graph showing the % suppression of extracellular HIV-1 p24 at various TG membrane protein concentrations.

FIG. 1C is a graph showing the % suppression of extracellular HIV-1 p24 at various treated and untreated TG membrane protein concentrations.

FIG. 2A is a graph showing the % suppression of extracellular HIV-1 p24 in 6000×g, 15000×g, and 60000×g supernatant pellets.

FIG. 2B is a graph showing the % suppression of extracellular HIV-1 p24 in TG supernatant pellets.

FIG. 2C is a graph showing the % suppression of extracellular HIV-1 p24 per sucrose density fraction.

FIG. 2D is a graph showing the % suppression of extracellular HIV-1 p24 in TG membrane and 15000×g supernatant pellet at 40% and 60% sucrose floatation.

FIG. 3A is a graph showing the % suppression of extracellular HIV-1 p24 in 33015 and 33074 cells.

FIG. 3B is a graph showing the % suppression of HIV-1 in exosome-enriched fraction, trypsin treatment and trypsin+chymotrypsinogen A treated samples.

FIG. 3C is a graph showing the % suppression of extracellular HIV-1 p24 in TG cells, untreated exosomes, methanol soluble fraction, precipitated protein, and chloroform fraction.

FIG. 3D is a graph showing the % suppression of extracellular HIV-1 p24 in untreated exosomes, delipidated insoluble proteins, delipidated soluble proteins.

FIG. 4 is a graph showing the % suppression of LTR-induced beta-galactosidase per exosome preincubation period prior to LTR induction.

FIG. 5 is a graph showing the % suppression of LTR-induced beta-galactosidase following virus-induced LTR activation, tat-induced LTR activation, and PMA-induced LTR activation.

FIG. 6 is a graph showing number of HIV-1 RNA copies over time.

FIG. 7A is a graph showing the % suppression of LTR-induced beta-galactosidase expression in TG exosomes and CD4+ exosomes.

FIG. 7B is a graph showing % suppression of LTR-induced beta-galactosidase expression in H9, Raji, U937, and Hela cells.

FIG. 8A is a graph showing the % suppression of LTR-induced beta-galactosidase expression in 6000×g depleted TG culture supernatant and 15000×g depleted TG culture supernatant.

FIG. 8B is a graph showing the % suppression of LTR-induced beta-galactosidase expression in 6000×g depleted TG culture supernatant and 15000×g depleted TG culture supernatant.

FIG. 8C is a graph showing the % suppression of LTR-induced beta-galactosidase expression in 6000×g depleted TG culture supernatant and 15000×g depleted TG culture supernatant.

FIG. 9A is a graph showing the % suppression of LTR-induced beta-galactosidase expression in purified CD8+ cells secreted exosomes, 6000×g depleted CD8+ cell culture supernatant, and 15000 depleted TG culture supernatant, in patient A.

FIG. 9B is a graph showing the % suppression of LTR-induced beta-galactosidase expression in purified CD8+ cells secreted exosomes, 6000×g depleted CD8+ cell culture supernatant, and 15000 depleted TG culture supernatant, in patient B.

FIG. 10A is a graph showing the % suppression of LTR-induced beta-galactosidase expression over time in exosomes from a TG culture maintained in log-phase growth.

FIG. 10B is a graph % suppression of LTR-induced beta-galactosidase expression over time in exosomes from a TG culture maintained at plateau phase.

FIG. 11 is a graph showing CD63 positive mean fluorescence shift per protein concentration of exosome sample dilution series.

FIG. 12A is a graph showing % suppression LTR-induced beta-galactosidase in three exosome samples.

FIG. 12B is a graph showing CD63 positive mean fluorescent shift in three exosome samples.

FIG. 13A is a graph showing the % suppression of LTR-induced beta-galactosidase expression in exosome-depleted TG supernatant and purified TG exosomes.

FIG. 13B is a graph showing the % suppression of LTR-induced beta-galactosidase in exosome-depleted TG supernatant and purified TG exosomes.

FIG. 13C is a graph showing the % suppression of LTR-induced beta-galactosidase in exosome-depleted TG supernatant and purified TG exosomes.

FIG. 14 is a schematic of a process of extraction of exosome soluble fractions according to one embodiment of the invention.

FIG. 15 is a graph showing the % suppression of LTR-induced beta-galactosidase expression in TG, untreated exosomes, storage buffer, and dialyzed sodium carbonate supernatant.

FIG. 16 is a schematic of a process of extraction of exosome soluble fractions according to one embodiment of the invention.

FIG. 17A is a graph showing the % suppression of LTR-induced beta-galactosidase in TG supernatant, storage buffer, NaCl supernatant, sodium carbonate supernatant first treatment, and sodium carbonate supernatant second treatment.

FIG. 17B is a graph showing % suppression of LTR-induced beta-galactosidase in untreated, sodium chloride treated, 1× sodium carbonate treated, and 2× sodium carbonate treated exosomes.

FIG. 18 is a graph showing the % suppression of LTR-induced beta-galactosidase in two samples each of untreated exosomes, step 1 ddH2O extraction, step 2 sodium carbonate extraction, and step 3 second ddH2O extraction.

FIG. 19A is a graph showing the % suppression of LTR-induced beta-galactosidase expression in supernatant fraction and exosome fraction in H9 exosome extractions and TG exosome extractions after step 1 ddH2O extraction and dialysis and after step 2 sodium carbonate extraction and dialysis.

FIG. 19B is a graph showing the % suppression of LTR-induced beta-galactosidase expression in step 1 sodium carbonate extraction and step 2 water extraction in H9 and TG exosome soluble protein extraction.

FIG. 20A is a graph showing the ratio of m/z 8.6 kDa to m/z 11.3 kDa peak integration areas for H9 and TG exosome ddH2O protein extraction by MALDI-TOF analysis.

FIG. 20B is a graph showing the % suppression of LTR-induced beta-galactosidase expression in H9 and TG exosome ddH2O protein extraction by LTR suppression assay.

FIG. 21 is a graph showing the % suppression of LTR-induced beta-galactosidase activity in an exosome source of ddH2O extracted sample of TG A, TG B, TG C, H9 A, H9 B, TG E, and TG D samples.

FIG. 22A is a graph showing the relative concentration of m/z 5.0 kDa corresponding protein in an exosome source of ddH2O extracted sample of TG A, TG B, TG C, H9 A, H9 B, TG E, and TG D samples.

FIG. 22B is a graph showing the relative concentration of m/z 5.4 kDa corresponding protein in an exosome source of ddH2O extracted sample of TG A, TG B, TG C, H9 A, H9 B, TG E, and TG D samples.

FIG. 22C is a graph showing the relative concentration of m/z 6.2 kDa corresponding protein in an exosome source of ddH2O extracted sample of TG A, TG B, TG C, H9 A, H9 B, TG E, and TG D samples.

FIG. 22D is a graph showing the relative concentration of m/z 8.6 kDa corresponding protein in an exosome source of ddH2O extracted sample of TG A, TG B, TG C, H9 A, H9 B, TG E, and TG D samples.

FIG. 23 is a graph showing the % suppression of LTR-induced beta-galactosidase expression in undialyzed ddH2O extracted sample and in a sample dialyzed through a 10 kDa cutoff filter.

FIG. 24 is a graph showing the % reduction after dialysis in relative m/z 5.0 kDa, m/z 5.4 kDa, m/z 6.2 kDa, m/z 8.6 kDa peaks compared to % reduction in LTR suppression activity.

FIG. 25 is a graph showing the % suppression of LTR-induced beta-galactosidase in storage buffer, NaCl, and sodium carbonate at high pH.

FIG. 26 is a graph showing the % suppression of LTR-induced beta-galactosidase expression at pH of 2.0, 3.0, 3.5, 4.0, 5.5, 7.0 and positive control.

FIG. 27 is a graph showing the % suppression of LTR-induced beta-galactosidase expression in samples with and without DDT at temperatures of 4, 47, 50 and 70 degrees C.

FIG. 28 is a graph showing the % suppression of LTR-induced beta-galactosidase expression in exosome bound and soluble extractions after a first, second and third extraction.

FIG. 29 is a schematic of a hypothetical model of protein interaction.

FIG. 30 is a graph showing retention of retroviral activity following retains its activity following lyophilization and reconstitution.

FIG. 31 is a graph showing sensitivity to trypsin and chymotrypsin.

### DETAILED DESCRIPTION OF THE INVENTION

In one embodiment, the invention provides an isolated or substantially purified antiretroviral polypeptide (which can include a peptide, fragment, analog or derivative thereof). By “antiretroviral,” in this context, it will be observed that the inventive polypeptide suppresses replication of a retrovirus. As used herein, the term retrovirus includes any virus belonging to the viral family Retroviridae, such as, for example, HIV-1, HIV-2, simian immunodeficiency virus (SIV), herpes virus saimir (HVS), and human T-cell leukemia virus (i.e., HTLV-I, HTLV-II, and HTLV-III). Importantly, the inventive antiretroviral polypeptide need not eliminate all retroviral replication—as inhibition will vary depending on the retrovirus and the assay in question.

The antiretroviral activity of the inventive polypeptide can be determined by assaying for the ability of the inventive antiretroviral polypeptide to suppress expression of retroviral long terminal repeat (LTR)-mediated genetic expression. For example, the inventive antiretroviral polypeptide can be identified as a polypeptide that suppresses HIV-1 LTR promoter expression by at least about 25% at a concentration of between about 1 ng/ml and about 10 ng/ml and typically about 95% suppression at concentrations between about 50 ng/ml and 100 ng/ml. This value can be determined according to an acute HIV-1 transcription suppression assay as described in Example 1 below. The inventive polypeptide can suppress the HIV LTR in the absence of HIV protein expression. Moreover, in some embodiments, the inventive compound also can suppress transcription from the LTR promoter of other retroviruses (e.g., HIV-2, SIV, FIV, HTLV).

In addition to being antiretroviral, the inventive polypeptide also is isolated or substantially purified. In this context, the protein exists in a cell-free preparation, and typically a serum-free preparation. More particularly, the inventive antiretroviral polypeptide is in a form in the absence of CD8+ and CD4+ T lymphocytes and B lymphocytes. Moreover, the inventive polypeptide is isolated from membrane fractions as well. Suitably, the inventive antiretroviral polypeptide exists in a preparation substantially isolated from other proteins or polypeptides, such as being at least 95% pure or at least 99% pure or at least 99.9% pure. For example, the antiretroviral polypeptide can exist within a composition consisting essentially of the antiretroviral polypeptide dissolved in water or a pH neutral aqueous buffer (e.g., Hanks, PBS) or in lyophilized form (which can contain a suitable cryopreservant (e.g., sucrose, trehalose), if desired).

The inventive antiretroviral polypeptide can be identified as a polypeptide having antiretroviral activity and also by the presence of at least one of the following characteristics: (a) a size less than about 13 kDa; (b) pH stable between about pH 4 through about pH 11.5; and (c) sensitive to trypsin. In some embodiments, the inventive polypeptide also can exhibit one or more additional properties: solubility in water; retained by a 5 kDa microfilter cassette; heat stable; derivable from CD8+ T lymphocytes, CD4+ T lymphocytes, B lymphocytes, or transformed cells thereof; derivable from a cell membrane, a cell surface, an endosomal compartment, a microvesicle, an exosome, or a combination of thereof; retaining anti-retroviral activity after lyophilization and resuspension; suppressing retroviral gene expression from an integrated long terminal repeat promoter; and sensitivity to chymotrypsin. In some embodiments, the inventive antiretroviral polypeptide possesses three or more of these qualities, such as four or more, five or more, six or more, seven or more, eight or more, or nine or more of these qualities. Suitably, the inventive antiretroviral polypeptide can be identified as possessing all of such qualities.

Solubility in water is believed to be attributed to the protein being substantially purified from water-insoluble lipid-rich cell fractions, such as membrane. Thus, water-soluble preparations of the inventive antiretroviral polypeptide are substantially free of lipids or membrane fractions. Water solubility can be assayed by extracting the protein with an aqueous system (which can include a suitable buffer, if desired). The aqueous extract then can be assayed for antiretroviral activity by measuring suppression of LTR promoter expression. Presence of antiretroviral activity in the aqueous fraction demonstrates that the protein is water soluble, which is consistent with the inventive antiretroviral polypeptide.

As noted, the inventive polypeptide can possess a size less than about 13 kDa. Mass spectroscopic techniques, such as electron spray ionization and matrix-assisted laser desorption time-of-flight (MALDI-TOF), can be used to determine the size of the inventive compound. In general, mass spectroscopy is an analytical technique used to measure the mass-to-charge (m/z) ratio of ions and is commonly used to find the composition of a physical sample by generating a mass spectrum representing the masses of sample components. Thus, the inventive antiretroviral polypeptide can contain one or more analyte signals as measured by mass spectroscopy. Exemplary mass spectroscopic signals indicative of the inventive antiretroviral polypeptide include m/z 8.6±0.1 kDa, m/z 6.2±0.1 kDa, m/z 5.4±0.1 kDa, m/z 5.0±0.1 kDa, m/z 2.5±0.1 kDa, and combinations thereof.

The size of the inventive antiretroviral polypeptide also can be identified by filtration techniques. For example, a membrane filtration (or ultrafiltration) process can be employed in which hydrostatic pressure forces a liquid against a semipermeable membrane and suspended solids and solutes of high molecular weight are retained, while water and low molecular weight solutes pass through the membrane. This separation process is used in industry and research for purifying and concentrating macromolecular (10³ to 10⁶ Da) solutions, especially protein solutions. Dialysis can be employed using desired cut-off filter sizes using standard microfilter cartridges (e.g., as manufactured by Millipore or Pierce). A solution containing the inventive protein can be tested for suppression of HIV-1 LTR promoter activity and then subjected to dialysis. Following dialysis, the solution can again be assayed for suppression of HIV-1 LTR promoter activity to determine whether and to what extent viral suppression is retained following dialysis or filtration or passes through the membrane/cartridge following dialysis or filtration. In this regard, the inventive antiretroviral polypeptide typically is retained by a 5 kDa cut-off microfilter cartridge. In some embodiments, the inventive antiretroviral polypeptide filters through a 10 kDa microfilter cartridge and in other embodiments, the inventive antiretroviral polypeptide does not filter through a 10 kDa microfilter cartridge. In this respect, dialysis using a 10 kDa microfilter cartridge can lead to retention of some HIV-1 LTR promoter suppression activity but also result in some HIV-1 LTR promoter suppression activity passing through the membrane.

By “heat stable” it is meant that the inventive antiretroviral polypeptide maintains at least about 95% of its antiretroviral activity (HIV-1 promoter suppression), preferably about 98% or more of its antiretroviral activity after heat application at 50° C. for five minutes. The inventive antiretroviral polypeptide further exhibits about 58% of its HIV-1 promoter suppression activity in the absence of DDT and about 35% in the presence of DDT upon heat application to 70° C. for five minutes. Heat stability can be assessed by warming a solution containing a polypeptide to a desired temperature for a suitable period of time (generally at least about 5 minutes), and then cooling the sample to about 37° C., after which it can be assayed for LTR promoter suppression activity.

pH stability can be determined by exposing the protein to differing pH conditions and assaying for its activity in suppressing HIV-1 LTR-mediated expression. In this regard, the inventive antiretroviral polypeptide exhibits low pH stability such that the inventive antiretroviral polypeptide retains at least about 70% of its antiretroviral activity when treated with an acidic solution having a pH of about 5.5 to about 7.0, and approximately 50% of its antiretroviral activity is retained when treated with an acidic solution having a pH of less than 5.5 but greater than 4.0. Further, the compound exhibits high pH stability such that the compound retains approximately 100% of its antiretroviral activity at a pH of from about 7.0 to about 11.5 relative to a control sample at pH 7.0. Example 3 herein, for example, reveals that the inventive protein exhibits about 70% suppression of HIV-1 LTR-induced expression at pH 11.5, which is about the same activity as observed either in storage buffer or NaCl at pH 7.

Typically, the inventive antiretroviral polypeptide is susceptible to inactivation upon treatment with trypsin and chymotrypsin. This can be assessed by exposing the soluble polypeptide to trypsin and/or chymotrypsin for a suitable time (e.g., about 6 hours) and under the appropriate buffer conditions for the enzymes, pelleted by centrifugation, washed and resuspended in media to assay for LTR promoter suppression activity.

Typically, the inventive antiretroviral polypeptide maintains antiretroviral activity following lyophilization and resuspension. This can be assessed by lyophilizing a preparation containing the inventive antiretroviral polypeptide and then resuspending it in water or a physiological pH buffered solution and then assaying for LTR promoter suppression activity.

In some embodiments, the inventive antiretroviral polypeptide can be obtained or derived from a CD8+ T lymphocyte, a CD4+ T lymphocytes, or B lymphocytes, or a transformed cell thereof. Typically, the inventive polypeptide is derived from a cell membrane, a cell surface, an endosomal compartment, a microvesicle, an exosome, or a combination of thereof. The inventive antiretroviral polypeptide can be derived from such sources by published methods or as described herein in the Examples. For instance, the inventive antiretroviral polypeptide may be extracted by delipidation of a cell membrane sample with a suitable organic solvent (e.g., chloroform/methanol, ethanol, ether, acetone, etc.), after which the precipitated proteins can then be harvested. Alternatively, an aqueous extraction from the surface of a cell membrane sample can be performed with a variety of salt, alkali, or pure water solutions. Detection using MALDI-TOF mass spectroscopic analysis of the fluid samples containing the soluble form of the inventive compound can then be used to isolate fractions, which can be assayed as described herein to identify the inventive antiretroviral polypeptide.

The invention further provides a pharmaceutical composition comprising the inventive compound. Preferably, the composition contains a pharmaceutically acceptable excipient, diluent, or carrier.

With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those ordinarily skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular method used to administer the compound. Accordingly, there are a variety of suitable formulations of the pharmaceutical composition. The following formulations for oral, aerosol, parenteral, subcutaneous, intravenous, intramuscular, interperitoneal, rectal, and vaginal administration are exemplary and are in no way limiting. One ordinarily skilled in the art will appreciate that these routes of administering the inventive compound are known, and, although more than one route can be used to administer the polypeptide, a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238 250 (1982), and ASHP Handbook on Injectable Drugs, Toissel, 4th ed., pages 622 630 (1986)).

Formulations suitable for oral administration can consist of (a) liquid solutions, such as an effective amount of the antagonist dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compositions can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non pressured preparations, such as in a nebulizer or an atomizer. Such spray formulations also may be used to spray mucosa.

Formulations suitable for parenteral administration include aqueous and non aqueous, isotonic sterile injection solutions, which can contain anti oxidants, buffers, bacteriostats, and solutes that render the formulation isotonic with the blood of the intended recipient, and aqueous and non aqueous sterile suspensions that can include suspending agents, solubilizers, thickening agents, stabilizers, and preservatives. The polypeptide of the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations are also well known to those of ordinary skill in the art. Such formulations are suitable in the context of the present invention for application to the skin.

Additionally, the compositions can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

Preferably, the compositions comprising the inventive compound are administered orally, or parenterally.

The invention also provides methods using the inventive antiretroviral polypeptide. In one embodiment, the invention provides a method of inhibiting viral replication within an infected cell. In accordance with the method, the inventive antiretroviral polypeptide is administered to the cell in an amount sufficient to inhibit the replication of the virus within the cell.

The inventive antiretroviral polypeptide can be administered to a cell in vitro or in vivo. Where the inventive antiretroviral polypeptide is administered in vivo, preferably it is admixed into a pharmaceutical composition. In such application, the invention provides a method of treating a subject (or patient) infected with a retrovirus. In accordance with the method, the subject is administered a therapeutically effective amount of a composition containing the inventive compound in an amount and at a location sufficient to treat the retroviral infection. In some embodiments, the method can result in remittance of the infection, while in other embodiments, the method can result in retardation of the progress of the infection. Either outcome, however, is therapeutically useful to the infected subject. Furthermore, the “subject” treated in accordance with the inventive method typically will be human, but the method also can be employed in the veterinary or laboratory context, in which the subject can be a non-human animal (e.g., a dog, a cat, a horse, a cow, a pig, a rat, a mouse, or a species of bird).

In yet another embodiment, a method of diagnosing an infection with a retrovirus is provided. In accordance with the method, a sample is taken from a subject (i.e., a human or animal), which is then assayed for the presence of the inventive antiretroviral polypeptide as described herein. The sample to be assayed can be any suitable tissue sample or fluid, but typically is blood or a blood product. Following assaying the sample, the presence of the inventive antiretroviral polypeptide can be correlated with an infection with a retrovirus in the subject.

In another aspect, the invention provides a method of extracting a peptide from an exosome. In one embodiment, the peptide is extracted from exosomes extracted therefrom by (a) purifying exosomes from cells; (b) adding storage buffer to the purified exosomes; (c) treating the exosomes with a high molarity salt solution; (d) pelleting the exosomes by centrifugation; and (e) extracting the supernatant from the treated exosomes, wherein the supernatant comprises soluble peptides. Preferably, the high molarity salt solution is a NaCl solution of about 1M (e.g., at least about 1M) concentration. Another high molarity salt solution suitable for removal of peripheral membrane proteins can be employed. However, the molarity of the salt solution should not be so high as to cause salt and/or protein precipitation.

In another embodiment, the method comprises (a) purifying exosomes from cells; (b) adding storage buffer to the purified exosomes; (c) pelleting the exosomes by centrifugation; (d) treating the centrifuged pellet of exosomes with a high pH solution; and (e) extracting the supernatant from the treated exosomes, wherein the supernatant comprises soluble peptides. The pH of the high pH solution is preferably greater than about 10, such as greater than about 11. For isolating the inventive antiretroviral polypeptide, a preferred solution is 0.1 M NaCOOH, pH 11.5, as the activity of the polypeptide is retained following such treatment.

In either of the above methods, the supernatant can thereafter be dialyzed into an aqueous pH neutral solution to collect the extracted polypeptides. Moreover, in either method, steps (b-e) may be repeated as can be the dialysis. Further, the exosomes may be derived from CD8+ T lymphocytes, CD4+ T lymphocytes, B lymphocytes, and transformed cells thereof or from other cells of interest.

In performing the inventive method, exosomes can be purified by any suitable technique. One method involves serial centrifugation of cell culture supernatant. For example, a 300×g spin can be used to remove cells, after which an 800×g spin can be used to remove large debris, a subsequent 6000×g spin can be used to remove microvesicles and other micron sized particles, followed by a final 15000×g spin to pellet the exosome fraction. The 15000×g pellet can then be subjected to sucrose gradient fractionation on a two layer 40%/60% discontinuous sucrose density gradient. The exosomes themselves then can be isolated in the band floating above the 60% sucrose cushion at the interface of the 40% and 60% sucrose layers.

Following purification, a storage buffer is added to the exosomes. Preferred buffers are pH neutral physiological buffers such as HANKS Balanced Salt Buffer (HBSS) or Phosphate Buffered Saline (PBS), which allow direct application of the extracted protein sample in a biological assay.

The method can be used to obtain the inventive antiretroviral polypeptide as well as other polypeptides from exosomes. For isolating the inventive antiretroviral polypeptide, exosomes can first be isolated by the sucrose gradient purification method described herein. Typically, a protein concentration estimate of the estimate is made by the Lowry or Bradford protein quantification methods. For extraction of the antiretroviral protein, purified exosomes are pelleted by centrifugation at 15000×g or higher. The supernatant is carefully removed and discarded.

The intact exosome pellet is then resuspended in either pure water, physiologically neutral buffer, 1M NaCl, or 0.1M Sodium Carbonate (pH 11.5) at a preferred final concentration between 1-2 mg/ml. For water or buffer extractions, the resuspended exosomes can be stored anywhere from 30 minutes to up to 24 hours to extract a soluble protein fraction containing the antiretroviral protein. For 1M NaCl or 0.1M Sodium Carbonate extractions, the resuspended exosomes are kept on ice for no more than 30 minutes.

After incubation with aqueous solution, the exosomes are centrifuged at 15000×g or higher. The supernatant is carefully extracted leaving the exosome pellet intact. In the case of water or buffer extractions, a small aliquot of sample from the extraction (10-20 microlitres) can be directly assayed for antiretroviral activity in a biological assay if the extraction was done from an exosome concentration of 1-2 mg/ml. If desired, the water or buffer extracted fractions containing the antiretroviral protein can be concentrated using a Millipore or Centricon centrifugation filter cartridge of 5 kDa molecular weight cutoff as described by the manufacturer (Millipore). In the case of treatment by 1M NaCl or 0.1 M Sodium Carbonate, after treatment and subsequent extraction of the aqueous supernatant upon pelleting the exosomes, the resulting salt solution must be dialized before testing the protein fraction for biological activity. Typically this dialysis is accomplished by using dialysis cassettes as manufactured by Pierce but the same can be effected using the Millipore or Centricon centrifugation filter cartridge of 5 kDa molecular weight cutoff. In either case, typically, a neutral pH buffer solution such as HBSS or PBS is preferred and dialysis is performed to enter the salt or alkali extracted fraction into a buffered solution suitable for biological assaying.

In most instances, extraction of a purified fraction containing the antiretroviral protein is desired, and towards this aim, a combination of serial aqueous treatments of a purified exosome sample can be performed. Typically, high molarity salts and high alkali treatments are performed to depleted peripheral proteins from the surface of exosomes that would otherwise contaminate preparations of the antiretroviral protein. After subsequent removal of peripheral proteins by high molarity salt or high alkali treatment, what remains are membrane proteins directly tethered to the lipid surface of exosomes. The soluble antiretroviral protein is derived by cleavage of a lipid tethered protein on the exosome surface. Thus, incubation of exosomes in pure water or buffer for up to 24 hours after removal of peripheral protein from the exosomes results in the extraction of the soluble antiretroviral protein from the exosomes.

The following examples further illustrate the invention but, of course, should not be construed as in any way limiting its scope.

### EXAMPLE 1

This example demonstrates that exosomes secrete an HIV-1 replication suppressing factor.

Cell lines and Virus Stocks. The transformation of primary CD8+ T cells with Herpesvirus Saimari (HVS) has been previously described in Chen et al., AIDS Res Hum Retroviruses, 16(2):117-24 (1993). An HVS-transformed CD8+ T cell clone, TG, was used, which was derived from primary CD8+ T cells purified from the peripheral mononuclear blood cells (PBMC) of an AIDS patient and transformed as previously described in Chen et al., Clin Diagn Lab Immunol., 4(1):4-10 (1997). Primary CD4+ T lymphocytes were selectively enriched as previously described in Chen et al., Clin Diagn Lab Immunol., 4(1):4-10, (1997), by immunomagnetic bead depletion of CD8+ cells from PBMC donated from an uninfected seronegative donor. Primary CD8+ T cells from two asymptomatic HIV-1 infected subjects were obtained through the Multicenter AIDS Cohort Study (MACS) at the University of Pittsburgh. The TZM-b1 cell line was obtained through the NIH AIDS Research and Reference Reagent Program, Division of AIDS, NIAID, NIH from Dr. John C. Kappes, Dr. Xiaoyun Wu, and Transzyme, Inc. The 8E5 cell line was obtained through the NIH AIDS Research and Reference Reagent Program, Division of AIDS, NIAID, NIH from Dr. Thomas Folks. TG, 8E5, primary CD4+ and CD8+ T cells were cultured in growth medium consisting of 20% FCS/RPMI supplemented with 25 mM HEPES and penicillin/streptomycin. TG cells and primary CD4+ and CD8+ T cells were supplemented with 5 U/ml of recombinant IL-2 (Roche, US). TZM-b1 cells were cultured in 10% FCS/DMEM supplemented with penicillin/streptomycin. M-tropic (R5) HIV-1 isolate 33015 was derived from an HIV-1 infected long-term nonprogressor patient from the MACS. The T-tropic (X4) HIV-1 isolate 33074 was obtained from an HIV-1 infected rapid progressor patient from the MACS. Immunomagnetic beads (Dynal, Norway) were utilized for cell separation (anti-CD8 beads) and exosome phenotyping (anti-MHC Class II beads). For exosome phenotyping by flow cytometry, fluorescently-labelled monoclonal anti-CD9, anti-CD63, anti-CD81, anti-CD14, and anti-CD34 and control isotype mouse IgG1 antibodies (Research Diagnostics Inc., US) were utilized.

Semi-quantitative Acute Infectious Suppression Assay. Suppression of acute HIV-1 infection was assayed using a semi-quantitative acute infectious suppression assay essentially as described by Chen et al., Clin Diagn Lab Immunol., 4(1):4-10 (1997). Peripheral blood mononuclear cells were isolated from an uninfected seronegative subject by ficoll-hypaque. Anti-CD8 antibody coated immunomagnetic beads (Dynal, Norway) were used for the separation of CD8+ and CD8− populations. CD8-depleted cells were cultured for 6 days in the presence of OKT3 and rIL-2 to expand and enrich for CD4+ T cells. After stimulation, cells were pretreated for 1 hour with 5 μg/ml polybrene, washed, and incubated with either HIV-1 R5-tropic 33015 strain or X4-tropic 33074 strain of HIV-1 for 2 hrs. Cells were washed after infection and subsequently cultured for 2 days in 20% FCS/RPMI with rIL-2, upon which, cells were DMSO-cryopreserved for use as target cells in an acute infectious suppression assay. A standardized protocol for measuring the HIV-1 suppression activity of a sample was performed by thawing the cryopreserved HIV-1 infected CD4+ cells and coincubation of TG cells or a derived sample. HIV-1 suppression activity of the sample was measured five days later as the percent reduction in extracellular p24 gag production, as measured by ELISA of culture fluid. This assay has demonstrated a high degree of standardization and reproducibility; Chen et al., Clin Diagn Lab Immunol., 4(1):4-10 (1997); Chen et al., AIDS Res Hum Retroviruses, 16(2):117-24 (2000).

Preparation of cell membrane. TG cells were harvested from culture and cell pellets were made of 100 to 500 million cells over the course of TG cell culture and stored at −70° C. until preparation of the membrane. Frozen pellets were thawed, resuspended into STM solution (sucrose, tris-HCl, MgCl₂), and subjected to three additional freeze-thaw cycles using ethanol dry ice for freezing and thawing in a 37° C. water bath. The disrupted cell suspension was homogenized using a Deunce homogenizer and the homogenate was clarified by centrifugation at 800×g, 4° C. to remove large cellular debris. Supernatant from this spin was then subjected to ultracentrifugation at 60,000×g for 30 minutes to pellet raw cell membranes. The pellet was then resuspended, overlayed on a 75% sucrose density cushion, and recentrifuged at 90,000×g/4° C. The band above the 75% sucrose interface was extracted, washed in STM buffer, re-pelleted by centrifugation, and resuspended in HANKS Balanced Salt Buffer or RPMI. Protein concentration was measured using the BioRad assay (BioRad, Hercules, Calif.).

Purification of exosomes. Exosomes and other membrane fractions were harvested from culture supernatants by an adaptation of methods previously described in Raposo et al., J Exp Med., 183(3):1161-72 (1996); Heijnen et al., Blood.94(11):3791-9 (1999), involving serial centrifugation of culture supernatant followed by sucrose density gradient purification. Conditioned culture fluid from TG cell cultures was harvested and first subjected to a 10 minute centrifugation at 300×g to remove cells. The supernatant was then subjected to serial centrifugations of increasing force to derive supernatants and pellets at 800×g for 30 minutes, 6,000×g for 30 minutes, 15000×g for 30 minutes, and 60,000×g for 60 minutes with all spins performed at 4° C. In such a manner, secreted membrane vesicles are derived at each centrifugation step with smaller debris pelleted at increased centrifugal force. As exosomes typically pellet at centrifugal force >10,000×g, Raposo et al., J Exp Med., 183(3):1161-72 (1996), the 15,000×g pellet was utilized for harvesting exosomes to avoid possible contamination with serum protein complexes in the culture media. A discontinuous sucrose density gradient separation was employed consisting of fractionation of the 15,000×g membrane pellet through a two layer sucrose column consisting of a 40% sucrose (1.14 g/ml) layer over a 60% sucrose (1.21 g/ml) cushion at 4° C. After centrifugation at 28,000×g/4° C., membrane fractions banded over the 40% and 60% sucrose interfaces and were extracted for further analysis and confirmation of exosome isolation in the 60% sucrose density fraction. Sucrose fractions were washed in HANKS buffer, pelleted by centrifugation at 18,000×g and resuspended in HANKS buffer. Protein concentration was measured using the BioRad assay (BioRad, Hercules, Calif.). For other cell lines in this study, such as primary CD4+ T cells, H9, Raji, 293T, and HeLa, exosomes were prepared from culture fluids from these cells essentially the same way they were prepared from TG cells.

Transmission Electron Microscopy. Copper grids (200 mesh) were formvar coated using 0.125% formvar in chloroform and floated on a drop of a highly concentrated exosome sample for approximately 30 seconds. The grids were removed and excess sample solution was wicked away with filter paper, then placed on a drop of 0.45 μm filtered 1% uranyl acetate in Milli-Q H₂O for 30-60 seconds. Excess stain was wicked away and samples were viewed on a JEOL JEM 1210 transmission electron microscope at 80 kV. Exosomes that were attached to Immunomagnetic Dynal beads (Dynal, Norway) were pelleted at 500×g in a 1.5 ml microfuge tube and fixed in 2.5% glutaraldehyde in PBS for 1 hr. Pellets were washed three times in PBS then post-fixed in 1% OsO₄, 1% K₃FE(CN)₆ for 1 hour. Following 3 additional PBS washes, the pellets were dehydrated through a graded series of 30-100% ethanol then infiltrated in Polybed 812 epoxy resin (Polysciences Inc, Warrington, Pa.) for 1 hr. After several changes of 100% resin over 24 hrs, pellets were embedded in a final change of resin, cured at 37° C. overnight, followed by additional hardening at 65° C. for two or more days. Ultrathin (70 nm) sections were collected on 200 mesh copper grids, and stained with 2% uranyl acetate in 50% methanol for 10 minutes followed by 1% lead citrate for 7 minutes. Sections were viewed using a JEOL JEM 1210 transmission electron microscope at 80 kV.

Flow Cytometry Analysis of Exosomes. Flow Cytometry analysis of exosomes was adapted from methods previously described by Clayton et al., J Immunol Methods, 247(1-2):163-74 (2001). Anti-MHC Class II antibody coated immunomagnetic beads (Dynal, Norway) were used to capture exosomes by incubation of high concentration vesicle sample (as determined by protein concentration) with 2.5×10⁵. Bead-captured vesicles were washed twice in cold buffer (4% FCS/PBS) and incubated with 10 μg/ml of anti-CD9, anti-CD63, anti-CD81, anti-CD14, anti-CD34, or isotype control biotinylated mouse IgG1 monoclonal antibody (R&D systems, Minneapolis, Minn.) for 30 minutes at room temperature. Beads were washed twice in cold buffer and incubated for 15 minutes room temperature with 1:50 diluted straptavidin-Phycoerythrin conjugate (Invitrogen, Carlsbad, Calif.). After a third round of washing, beads were fixed in 1% paraformaldehyde and analyzed on a Beckman Coulter EPICS XL.MCL Flow Cytometer.

Protease Treatment. Aliquots containing 60 μg of TG exosome were pelleted by centrifugation at 17,000×g and resuspended in 1 ml of 5 mM Trypsin solution, or 1 ml of 5 mM Trypsin+5 mM Chymotrypsinogen A. Control exosomes were resuspended in HANKS buffer. Protease treatments and controls were incubated at 37° C. for 6 hours. Protease-treated exosomes and controls were then pelleted by centrifugation, washed with HANKS buffer and resuspended in 300 μl of culture media (20% FCS/RPMI).

Delipidation of Exosomes. Exosomes were pelleted by centrifugation. In one experiment, delipidation of exosomes was performed as described by (Bligh and Dyer, Can. J. Biochem. Physiol. 37:911-917 (1959)). Pelleted exosomes were resuspended in a 2:1 mixture of chloroform/methanol, resulting in extraction of lipids into chloroform phase, and proteins extracting into methanol solution and an insoluble precipitate at the chloroform methanol interface. The three fractions were extracted and dried for further analysis. In a second delipidation method, cold acetone (−20° C.) was used to dissolve exosomes and precipitate membrane protein. Precipitated proteins were resuspended into RPMI, centrifuged for 5 minutes at 17,000×g to separate undissolved proteins from those remaining in solution. After acetone delipidation, undissolved and dissolved proteins were analyzed for HIV-1 suppression activity.

Acute HIV-1 Transcription suppression assay. An assay for measurement of LTR promoter inhibition in a model mimicking acute infection was adapted from the methods of Chang et al., J Virol. 76(2):569-81 (2002). TZM-b1 cells were seeded 25,000 cells/well and cultured at 37° C. for 24 hrs. TZM-b1 cells were then incubated with TG exosomes or culture fluid sample for 16-24 hrs at 37° C. Cells were washed twice with media prior to LTR activation. For gene-reporter expression induced by virus infection, TZM-b1 cells were inoculated with HIV-1 primary isolate 33015 and supplemented with 8 μg/ml DEAE-dextran for 1 hour, washed with media and incubated at 37° C. for 24 hrs after infection. For tat-transactivated LTR induction, TZM-b1 cells were liposome-transfected with the tat-expressing plasmid pSVtat using the LIPOFECTAMINE 2000 reagent according to the manufacturer's instructions (Invitrogen, Carlsbad, Calif.). For mitogen-activation of the LTR promoter, TZM-b1 cells were incubated with 100 ng/ml PMA (Invitrogen, Carlsbad, Calif.) for 12 hours. The extent of LTR-induced gene expression of β-galactosidase was measured using the β-GLO Assay (Promega, Madison, Wis.).

Chronic HIV-1 Transcription suppression assay. 8E5 cells were incubated in the presence or absence of TG exosomes over a time course of 25 days. Cell numbers were maintained between 5,000 and 50,000 cells per well in a 96 well plate and cell numbers were adjusted every 5-7 days with replenishment of media alone or media supplemented with TG exosomes. At each 5-7 day time point, 1000 cells were collected and carefully measured to assay intracellular HIV-1 RNA copies per 1000 cells using the NASBA method (Organon Teknika, Dublin, Ireland).

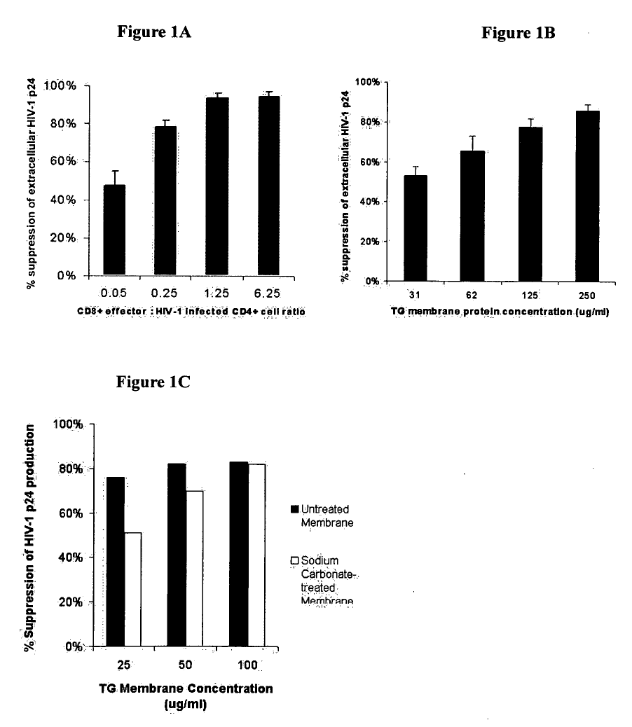

Results. Membrane from the CD8+ T cell line TG, suppresses HIV-1 replication. While CD8+ T cell noncytolytic suppression of HIV-1 has been previously described as mediated by soluble factors, experiments in which CD8+ T cells and HIV-1 infected CD4+ cells are separated by a semi-permeable membrane demonstrate that this antiretroviral mechanism is most efficient with cell to cell contact. Therefore, to explore whether membrane protein derived from CD8+ T cells could suppress HIV-1 to a similar extent observed with cell mediated suppression, the TG CD8+ T cell line was cultured in a large quantity for cell membrane purification. The TG cell line contained potent dose-dependent HIV-1 suppression activity against acutely infected primary CD4+ T cells (FIG. 1A). Membrane from this cell line was purified and it was found that it could by itself mediate the same dose-dependent HIV-1 suppressive effect in acute infection assay (FIG. 1B). Since a secreted factor has been described as one of the defining characteristics of noncytolytic HIV-1 suppression activity by CD8+ T cells, the next step was to discern if the TG membrane-mediated HIV-1 suppression activity was due simply to a peripheral membrane protein. Therefore, TG membrane was treated with 0.1 M sodium carbonate at pH 11.5 to deplete peripheral proteins from the membrane. After treatment, membrane was pelleted by centrifugation at 17,000×g, washed, resuspended in media, and assayed alongside an untreated control for dose-dependent HIV-1 suppression activity. Only a moderate decrease in HIV-1 suppression activity was detected after sodium carbonate treatment indicating that the majority of the activity specifically resided in the membrane, indicating the presence of some membrane localized factor(s) capable of suppressing HIV-1 replication (FIG. 1C).

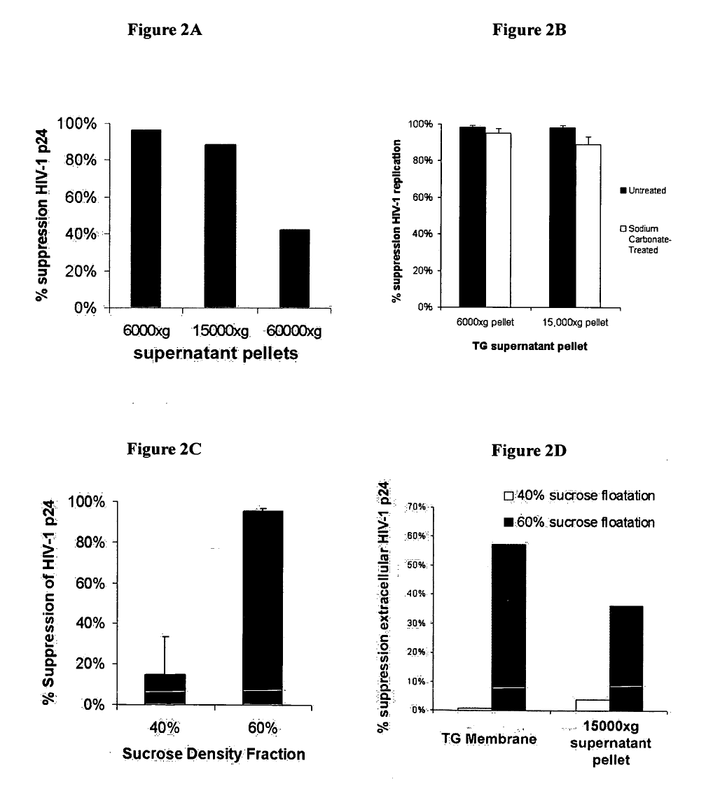

To account for the membrane-bound nature of the antiretroviral activity and its reported appearance in soluble form, it was hypothesized that this activity might be secreted in a vesicular form by CD8+ T cells. It was reasoned that if the cell surface contained HIV-1 suppressive activity, then vesicles secreted by the TG cells would likely carry at least some of the same membrane determinants from the cell surface and this may make some contribution. The secreted vesicles reported in the literature have been described as two general types: (i) 1 uM sized microvesicles originating from the plasma membrane and (ii) 30-100 nm sized exosomes originating intracellularly from endosomal compartments, Heijnen et al., Blood, 94(11):3791-9 (1999). Therefore, the TG cell line was tested to see if it might also be secreting similar vesicles containing HIV-1 suppressive activity. Conditioned media from the TG cell cultures was subjected to increasing serial centrifugation to derive membrane pellets of decreasing size. In such a manner, fractions of 6000×g, 15000×g, and 60000×g were collected from cell-free culture media of TG cells and standardized by volume. These fractions were assayed for suppression activity using the acute infectious suppression assay, and indeed found potent HIV-1 suppression activity peaking at 6000×g and 15000×g membrane fractions (FIG. 2A). To verify whether these peak TG culture supernatant membrane fractions also maintained the same property of membrane-localization of HIV-1 suppression activity that bulk TG membrane maintained after removal of peripheral proteins, the 6000×g and 15000×g fractions were treated with 0.1 M sodium carbonate in the same manner as for bulk membrane, and found only a slight diminishment of activity after treatment in either pellet (FIG. 2B). This further suggested the existence of a membrane localized factor mediating HIV-1 antiretroviral activity.

As the secreted vesicles clearly had a tightly bound HIV-1 suppressive activity, the origin was sought in order to further determine their functional nature to contact-dependent noncytolytic CD8+ T cell HIV-1 suppression activity. A good candidate for such vesicles appeared to be exosomes as they typically pellet at centrifugal force greater than 10,000×g, Raposo et al., J Exp Med., 183(3):1161-72 (1996); Heijnen, et al., Blood, 94(11):3791-9 (1999). Therefore, the 15000×g fraction was applied onto a discontinuous sucrose gradient consisting of a layer of 40% sucrose over a 60% sucrose cushion. The sucrose gradient was based on previous methods, which demonstrated exosomes being consistently harvested within a 1.14-1.21 sucrose density gradient in Raposo et al., J Exp Med., 183(3):1161-72 (1996); Heijnen, et al., Blood, 94(11):3791-9 (1999). After fractionating the 15000×g sample, two distinct bands were harvested, one floating above the 40% sucrose layer representing vesicle densities of 1.0-1.14 g/ml and a second band above 60% sucrose interface representing vesicle densities in the 1.14-1.21 g/ml range. After washing and pelleting the two fractions, they were resuspended and standardized to equivalent protein concentration. The two fractions were assayed for HIV-1 suppression activity in the acute infection assay and found that potent HIV-1 suppression activity was contained in the 1.14-1.21 g/ml fraction that floated at the 60% sucrose density interface (FIG. 2C). After preparation of several other samples, it was noticed that HIV-1 suppression activity consistently peaked with the 60% sucrose fractions. In fact, when the same sucrose density gradient fractionation was applied to a purified TG membrane sample, HIV-1 suppressive activity was localized specifically to the 60% cell membrane fraction as it did for the 60% secreted vesicle fraction (FIG. 2D).

Identification of HIV-1 suppressing TG vesicles as exosomes. The specific localization of HIV-1 suppression activity to 60% sucrose density fractions is significant as it corresponded to the sucrose densities previously reported for exosomes secreted by other cell types, Wubbolts et al., J Biol. Chem., 278(13): 10963-72 (2003); Escola et al., J Biol. Chem. 273(32):20121-7 (1998), Raposo et al., J Exp Med., 183(3): 1161-72 (1996), Thery et al., Nat. Rev. Immunol. 2, 569-579 (2002). Therefore, the next step was to elucidate the identity of these TG secreted particles. A fresh 15000×g/60% TG supernatant vesicle sample was prepared for analysis by transmission electron microscopy (TEM). TEM revealed the highly enriched presence of vesicles resembling the 30-100 nm size and spherical morphology of exosomes, as previously described for a variety of other cell types.

In order to confirm the identity of the TG vesicles as exosomes, a recently described exosome bead-capture technique (Clayton et al., J Immunol Methods, 247(1-2): 163-74 (2001)) was used that is based on the enriched presence of MHC Class II molecules on the endosomally derived vesicles. The bead-capture technique utilizes immunomagnetic beads coated with antibodies specific for MHC Class II molecules. By coating the surface of the 4.5 μm diameter spherical beads with nanovesicles, their antigenic content can be probed to confirm their presence as exosome markers. A high concentration sample of the 15000×g/60% vesicle fraction was incubated with the immunomagnetic beads at 4° C. overnight, after which the beads were magnetically separated and washed. Two aliquots of beads after vesicle incubation were made, one for electron microscopy analysis to confirm bead capture and the second aliquot for determining the antigenic content by flow cytometry. The bead surface was analyzed by ultrathin section electron microscopy and it was found that the perimeter of bead surfaces were indeed saturated with the tiny vesicles, confirming their attachment to the beads. Concurrently, the same aliquot of bead-captured vesicles which were prepared for TEM analysis were analyzed to detect their antigenic content by flow cytometry, using specific monoclonal antibodies to probe for the presence of exosome-specific markers. Detected was the specific presence of CD9, CD63, and CD8 on the TG vesicle coupled beads with CD63 producing the highest fluorescence shifts. CD14 was not detected while moderate amounts of CD34 were observed. In addition, antibody staining of control beads did not produce any fluorescence shift in control experiments thereby indicating that the fluorescence shift relative to isotype control detected for CD9, CD63, and CD81 were specifically due to the presence of the vesicles attached to the beads. CD9, CD63, and CD81 belong to the tetraspanin family of proteins and are highly enriched in exosomes from a variety of cell types, Thery et al., J Immunol., 166(12):7309-18 (2001); van Niel et al., Gastroenterology, 121(2):337-49 (2001). Additionally, CD63 is a specific lysosomal marker that also traffics to endosomal compartments Mahmudi-Azer et al., Blood, 99(11):4039-47 (2002); Pfistershammer et al., J Immunol., 173(10):6000-8. (2004), so their high expression on the vesicles relative to other markers indicates their specific endosomal origin. Thus, the combined tetraspanin enrichment, endosomal origin, density in sucrose, size and morphology of these vesicles specifically identify them as TG cell-secreted exosomes with potent HIV-1 suppressive activity.

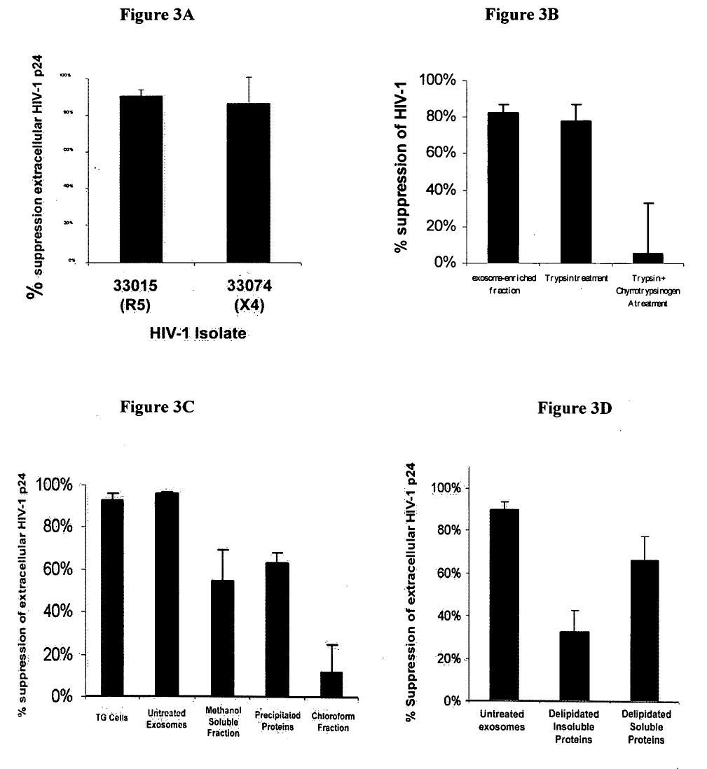

TG exosome suppression of R5 and X4 isolates is protein mediated. A hallmark of noncytolytic CD8+ T cell suppression of HIV-1 is the inhibition of CCR5-tropic and CXCRX4-tropic HIV-1 replication, Chang et al., J Virol., 76(2):569-81 (2002). Therefore, TG exosomes were assayed for their ability to suppress two patient derived HIV-1 isolates: (i) 33015, an R5 clinical isolate and (ii) 33074, an X4 clinical isolate. Using the acute infectious suppression assay it was found that TG exosomes could suppress the replication both R5 and X4 HIV-1 isolates (FIG. 3A). In order to confirm that the action was specifically due to a protein factor on the exosomes, separate exosome samples were either untreated, treatment with trypsin, or a combination of trypsin and chymotrypsinogen A for 6 hours, pelleted by centrifugation, washed and resuspended in media to assay for HIV-1 suppression activity. Exosome treatment with trypsin alone did not weaken the exosome-mediated HIV-1 suppressive activity, however, treatment with a combination of trypsin and chymotrypsinogen A abrogated the antiretroviral activity (FIG. 3B).

The proteolytic inactivation of exosome-mediated HIV-1 suppression activity indicated that the active domain of the putative factor mediating the antiretroviral activity is expressed ectopically on the surface of the TG exosomes. To further corroborate the specific involvement of such a protein, a series of membrane delipidation experiments were performed to determine if a protein mediator could be extracted into solution from the exosomes. Such experiments were crucial to determining whether a hypothetical exosome fusion mechanism was involved and to rule out a possible nonspecific lipid inhibition of HIV-1 replication.

Therefore, exosome delipidation was performed using 2:1 chlorform/methanol, which extracts lipids into the chloroform phase, and proteins into the methanol phase and as precipitates at the chloroform-methanol interface (Bligh and Dyer, Can. J. Biochem. Physiol. 37:911-917 (1959)). After subjecting TG exosomes to the treatment, the methanol-phase, precipitated proteins, and chloroform fraction were extracted and dried using a speedvac. The three fractions were resuspended in media and assayed for HIV-1 suppression activity. It was found that HIV-1 suppression activity was specifically localized to the precipitated proteins and the methanol soluble protein fraction but not to the chloroform phase, indicating that the lipid moiety of exosomes was not involved in mediating HIV-1 suppression (FIG. 3C). To further confirm this result, another delipidation experiment was performed, this time using cold acetone to deplete exosome lipids. In this method, lipids are extracted into the organic phase producing a protein precipitate. Upon resuspension of the acetone precipitate protein, it was found that not all the protein entered into solution, so further separation was conducted of the insoluble protein from those that remained soluble, and both were assayed for HIV-1 suppression activity with the insoluble protein fraction that was added in as a mixture. While a small amount of HIV-1 suppression activity was detected in the mixture, most of the acetone-precipitated protein activity resided in the soluble protein fraction (FIG. 3D). Thus, the results of these delipidation experiments corroborated results of the previous proteolytic inactivation experiment, demonstrating that exosome suppression of HIV-1 was specifically mediated by a protein expressed on the extracellular surface of exosomes. Furthermore, the non-involvement of lipid was additionally confirmed upon numerous observations that TG exosomes maintained intact antiretroviral activity even after multiple freeze-thaw cycles as well as sonication. Together, these results indicated that the protein mediated antiretroviral activity was exerted irrespective of its membrane localization.

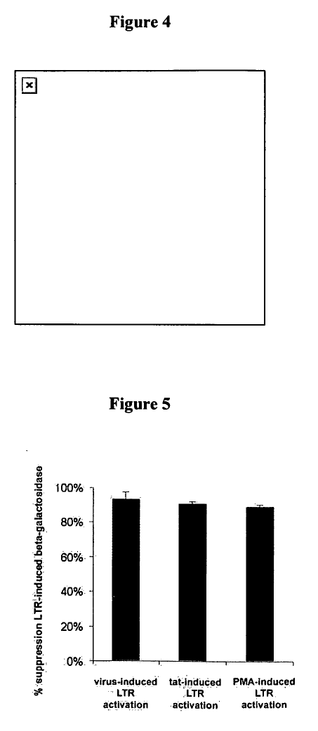

TG exosome suppression of HIV-1 transcription. To further determine the nature of the suppressive activity localized to the TG exosomes, the next step was to determine whether the antiretroviral activity specifically inhibited HIV-1 at the level of its proviral transcription. First, HIV-1 promoter suppression activity was assessed in an LTR-activated gene-reporter assay that essentially mimics an acute infection model. The HeLa derived TZM-b1 cell line that has been genetically engineered for stable expression of CD4 and CCR5, Rubinstein et al., Eur J Immunol, 26(11):2657-65 (1996) was utilized. Furthermore, this cell line also contains two stably integrated LTR-reporter genes consisting of one construct with the 5′LTR fused to the β-galactosidase gene and a second construct with the 5′LTR fused to a luciferase gene. Expression of the gene-reporters can be activated in the cell line by HIV-1 infection, transfection of a tat-expressing plasmid, or by mitogen stimulation by PMA. The implementation of this cell line was based on the methods of Chang et al., J Virol., 76(2):569-81 (2002). In adapting the TZM-b1 cell line for assaying acute LTR suppression, a titration was performed by preincubating TZM-b1 cells with TG exosomes for 3, 6, 12, or 24 hours prior to LTR induction of gene reporter by HIV-1 inoculation. After LTR induction, cells were cultured for 24 hrs upon which, intracellular β-galactosidase was assayed. It was found that maximum suppression of β-galactosidase occurred only when exosomes were preincubated with TZM-b1 cells for at least 6 hours (FIG. 4). To confirm that the exosome-induced block in β-galactosidase expression was specifically due to HIV-1 LTR promoter repression, TZM-b1 cells were pre-incubated with TG exosomes for 12 hours, upon which β-galactosidase expression was activated by either virus inoculation, liposome-transfection with the tat expressing pSVtat plasmid, or mitogen activation with 100 ng/ml PMA. After 24 hour post-induction incubation of TZM-b1 cells, it was found that TG exosomes mediated potent suppression of the LTR promoter regardless of whether it was virus-, tat-, or PMA-induced (FIG. 5). This further demonstrates that HIV protein expression is not required for the activity of the inventive protein.

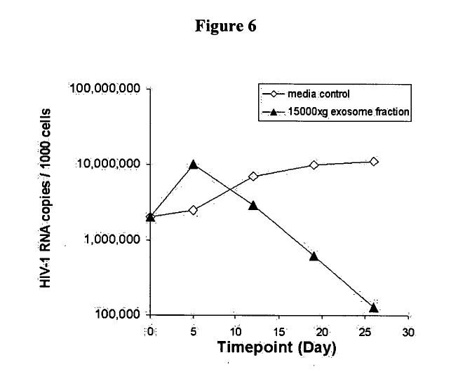

Since the LTR gene-reporter assay mimicked an acute infection model, a determination was sought of whether the CD8+ cell-secreted exosomes were also capable of suppressing HIV-1 transcription in a chronic model of infection. Toward this aim, the chronically-infected 8E5 CD4-negative T cell line, Folks et al., J. Exp. Med., 164, 280-290 (1986) was used as a target to assay exosome-mediated HIV-1 transcriptional repression. 8E5 cells contain a single full-length copy of an integrated HIV-1 LAV genome with a null mutation in its reverse transcriptase that results in the production of non-infectious virions, Folks et al., J. Exp. Med., 164, 280-290 (1986). Since no cell-to-cell transmission of virus occurs, any suppression of HIV-1 in the 8E5 cell line is specifically directed at a post-integration step of the virus life cycle. 8E5 cells were cultured in the absence or presence of purified TG exosomes in a time course experiment. Total HIV-1 RNA copies per 1000 cells were measured every 5-7 days and cells were replenished at each time point with media alone or media supplemented with TG exosomes in addition to adjusting cell concentrations to maintain healthy cell growth. After measuring an initial transient spike in HIV-1 RNA at day 5 in 8E5 cells cultured in the presence of exosomes, it was subsequently noted that a dramatic and sustained exosome-induced reduction of intracellular HIV-1 transcripts that were not observed for controls (FIG. 6).

A decrease of more than 2 Log10 in HIV-1 transcripts was observed for 8E5 cells cultured in the presence of exosomes compared to controls at the last time point. The reduction of HIV-1 RNA only after Day 5 for 8E5 cells cultured in the presence of exosomes is consistent with a delayed kinetics in LTR promoter induction as demonstrated in the TZM-b1 cell line (FIG. 4). The potent HIV-1 transcription suppression in acute and chronic models clearly defines the mechanism TG exosomes employ to suppress HIV-1 replication.

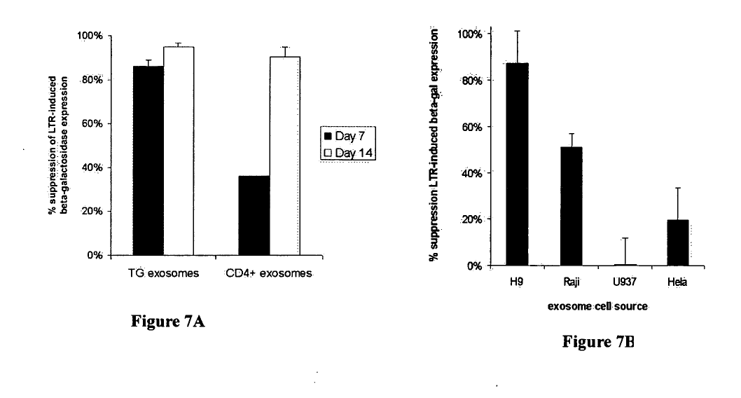

Cell specificity of exosome-mediated suppression of HIV-1 transcription. The results thus far indicate that TG exosomes correlate with key hallmarks defining noncytolytic CD8+ T cell suppression of HIV-1, namely the suppression of R5 and X4 HIV-1 isolates and specific inhibition of the viral LTR promoter in acute and chronic models of infection. A determination was sought as to whether the TG exosomes might satisfy a third hallmark of the antiretroviral activity—specificity to CD8+ T cells. Several studies have noted that cell-mediated noncytolytic HIV-1 suppression appears to be an exclusive function of CD8+ T cells, Levy, Trends Immunol., 24(12):628-32 (2003). Thus, a corollary supposition would be that membrane determinants mediating cell-contact dependent HIV-1 suppression would be cell specific. This possibility was studied by comparing the HIV-1 transcription suppression activity of TG exosomes to exosomes secreted by other cell types. In the initial analysis primary CD4+ T cells were collected from a seronegative donor and activated with OKT3 anti-CD3 antibody and recombinant IL-2 for 7 days. At Day 0 of the CD4+ T cell culture, an independent parallel TG cell culture was separated into fresh media so that at day 7, exosomes were harvested from both TG and CD4+ T cell culture fluids and assayed for HIV-1 transcription suppression activity using the TZM-b1 gene-reporter assay. TG and CD4+ T cells were recultured, this time stimulating CD4+ T cells only with rIL-2. On Day 14, exosomes were prepared from the TG and CD4+ T cells and assayed for HIV-1 suppression activity. It was found that for exosomes from day 7 samples, TG exosomes suppressed the LTR to a 2.3-fold higher level than CD4+ cell derived exosomes (FIG. 7A Black Bars). However, at day 14, CD4+ cell exosomes were found at much higher levels of LTR suppressive activity now, comparable to the high suppressive activity maintained by the TG exosomes (FIG. 7A Black Bars). These initial results suggested that exosome mediated suppression of HIV-1 transcription was not necessarily exclusive for CD8+ T cells. To verify this, exosomes from several distinct cell lines were analyzed. Large cultures of H9, a CD4+ T cell line, Raji, an EBV-transformed B cell line; U937, a monocyte cell line, and the HeLa cell line were prepared. After culturing each cell line at sufficient volume and saturation, exosomes were harvested from culture fluids and assayed for HIV-1 transcription suppression. In support of previous results, it was found that H9 exosomes displayed potent LTR suppression activity, while moderate amounts of LTR suppression activity were deteded in Raji exosomes and no suppression detected in U937 exosomes and very little for HeLa (FIG. 7B). These results suggest that exosome mediated HIV-1 suppressive activity is not the exclusive domain of CD8+ T cells.

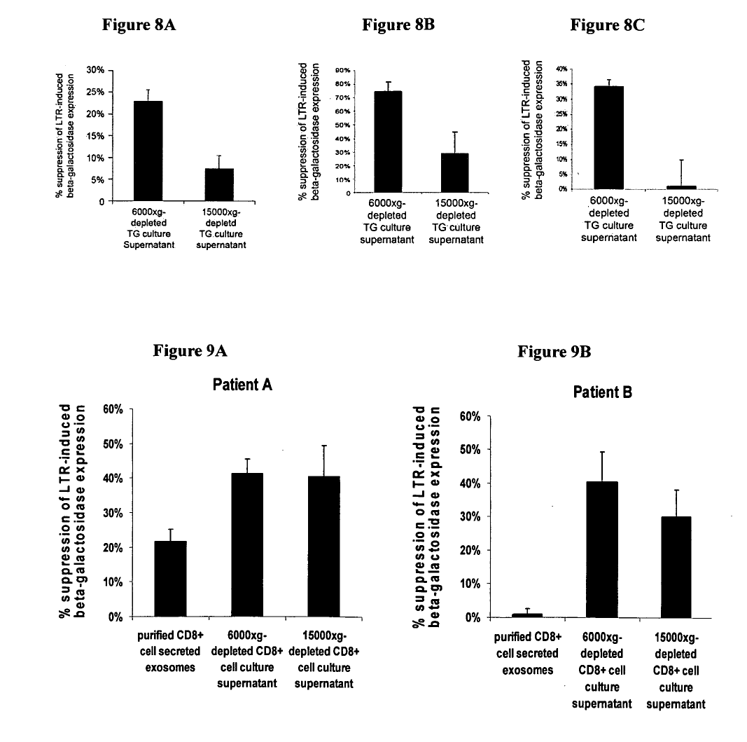

Contribution of exosomes to secreted antiretroviral polypeptide activity. Since TG exosomes exhibited potent HIV inhibition activity, an investigation was conducted to determine the contribution of exosomes in the context of their physiological release and contribution to antiretroviral polypeptide activity. It was observed that, in the original fractionation of membrane vesicles from TG culture fluids, only moderate amounts of antiretroviral activity was present in 60,000×g membrane pellets compared to 6000×g and 15000×g pellets (FIG. 2A). This would indicate that centrifugation at 15000×g removes a sizable amount of exosome-mediated activity from the TG supernatant. The question then becomes whether or not HIV-1 transcription suppression activity also diminishes accordingly in the exosome-depleted culture fluids. If a secreted factor is purely membrane bound then reduction of the vesicles expressing the factor should be coincident with a reduction of antiretroviral activity. However, if vesicles are depleted but a substantial portion of the activity still remains, it would indicate the presence of a soluble protein mediating the same activity. To address this issue, small samples of TG culture fluids were prepared from six independent TG cell cultures and after depleting the culture fluids of cells and large debris, the samples were subjected to serial centrifugation at 6000×g followed by a 15000×g spin, removing aliquots from each step for analysis of HIV-1 transcription suppression activity using the TZM-b1 assay. It was found that in three exosome samples LTR suppression activity reduced significantly after the 15000×g step (FIG. 8A-C). Although the reduction in antiretroviral activity appeared to be incomplete in two of the samples (FIGS. 8A and B), the results nonetheless demonstrated that the CD8+ cell secreted exosomes constituted a majority of the LTR suppression activity present in the culture fluid samples.

Next, the contribution of exosomes to antiretroviral polypeptide activity in other CD8+ cell culture fluids was determined. Blood samples from two asymptomatic HIV-1 infected patients were obtained and cultured their CD8+ T cells, first by activation with OKT3 anti-CD3 antibody and IL-2 for 5 days, after which cells were washed and re-cultured in fresh culture media supplemented with IL-2 for 5 days. Exosomes were purified from the culture fluid and were assayed for HIV-1 suppression activity along with aliquots of 6000>g- and 15000×g-depleted culture fluids (FIGS. 9A-B).

Surprisingly, the purified exosome samples were deficient in LTR suppression activity compared to TG exosomes and exosomes from CD4+ T cells and H9 cells, with one patient displaying only a small amount of activity (FIG. 9A) and the second displaying no exosome-mediated LTR suppression (FIG. 9B). Furthermore, exosome-depletion did not significantly reduce the extent of LTR suppression activity in 15000×g-depleted CD8+ cell culture fluids for either patient CD8+ T cell culture (FIGS. 9A and B). In this instance, the activity appeared to be mediated by a completely soluble factor with the exosomes containing little to no activity. Whereas in the TG culture fluids, exosomes were the dominant contributor to antiretroviral polypeptide activity. In fact, the contrasting results between the limited TG cell line and primary CD8+ cell culture fluid samples analyzed might actually be indicative of a functionally inverse correlation between an exosome-bound and membrane-free mediators of HIV-1 transcription inhibition. The data are consistent with the inventive polypeptide representing a soluble protein derived from an exosome-bound precursor.

### EXAMPLE 2

This example characterizes an LTR suppressing factor and demonstrates a novel technique to identify the factor.

TG cell cultures, exosome preparations, and the acute LTR suppression assay using the TZM-b1 gene-reporter cell line are used in this example as described in Example 1, with some minor modifications where noted. In addition, the TZM-b1 assay that was used throughout as this gene-reporter assay has been proven to be a very sensitive and reproducible assay for the evaluation of biochemically extracted samples mediating LTR promoter inhibition (Tumne and Gupta, Unpublished Data).

Exosome Preparation. Exosomes were prepared essentially as described in Example 1 by serial centrifugation of cell culture supernatant followed by sucrose gradient fractionation of the 15,000×g membrane pellet. In some experiments, after the final wash and pelleting of exosomes from the 60% sucrose density gradient fraction, exosomes were resuspended 0.1 M Sodium Carbonate instead of RANKS balanced salt buffer.

Quantitative Exosome Assay. The method of Clayton et al., J Immunol Methods. 247(1-2):163-74 (2001) was adapted to develop a quantitative assay for measurement of relative exosome concentrations between samples under nonsaturating conditions of exosome bead-capture. Immunomagnetic beads coated with polyclonal antibodies to MHC Class II (DYNAL, Norway), were washed and resuspended at a concentration of 5×10⁶/ml in 2% FCS/PBS. A volume of 200 ul containing 10⁶ beads was mixed with 50 ul of sample containing exosomes and incubated on a rotator (DYNAL, Norway) at 4° C. for 16 hrs at 35 rotations per minute. After bead-exosome incubation, beads were washed twice with 2% FCS/PBS and stained with PE-labelled monoclonal antibody to CD63 for analysis by flow cytometry, as described in Example 1. The extent of CD63-dependent fluorescence shift relative to isotype antibody controls, under conditions of non-saturating exosome-bead binding, is directly proportional to the concentration of exosome in the sample. A proof-of-principle for the technique was given by titration of an exosome dilution series from three independent exosome preparations in which the extent of CD63-dependent fluorescence shift correlated linearly with exosome concentration standardized by protein content (FIG. 11).

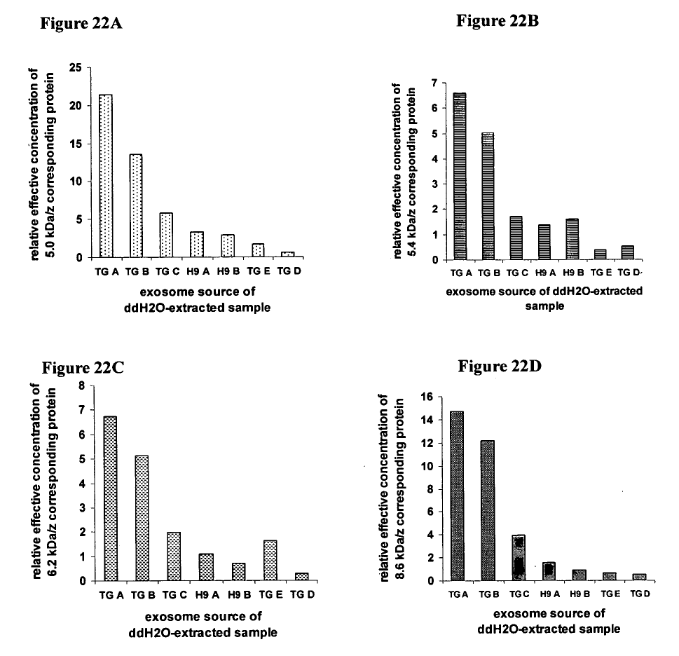

Extraction of Peripheral Membrane proteins from the exosomes. Exosomes were pelleted by microfuge centrifugation at 20,000×g for 30 minutes. Exosomes were then resuspended in a variety of solutions for extraction of peripheral membrane proteins at 4° C. These treatments included 1M NaCl for 30 minutes, HANKS Balanced Salt Buffer for 30 minutes, and storage at 4° C. or −70° C. of freshly prepared exosomes, 0.1 M sodium carbonate, pH 11.5 for 30 minutes, deionized double distilled water for 16-24 hrs. Upon treatment, exosomes were re-pelleted by microfuge centrifugation to extract supernatant containing peripheral proteins. Dialysis and concentration of extracted supernatant after salt treatments (sodium carbonate, sodium chloride, HANKS Buffer) was performed by three successive rounds of washing and microfiltration using a 5 kDa cutoff microfilter cartridge (Millipore, US). The final 5 kDa microfilter dialyzed concentrate was resuspended into media for assaying HIV-1 suppression activity at a volume equivalent to the original exosome preparation from which the extract was derived. Dialysis using the 5 kDa cutoff microfilter was found to fully retain LTR suppression activity. Dialysis of samples using a 10 kDa cut-off dialysis membrane cassette (Pierce, US) was found to retain most, but not all of the activity (FIG. 22A).

MALDI-TOF analysis of TG and H9 catalytically released proteins. TG and H9 secreted exosomes were purified, assessed for protein concentration using a BioRad assay, treated with 0.1 M Na₂CO₃ pH 11.5 to remove peripheral proteins from the exosomes. After a 30 minute treatment at 4 C, the membrane fraction was separated from the supernatant by centrifugation at 20,000×g. The resulting membrane was washed 3 times with de-ionized ddH2O (dI-ddH2O) to remove residual salt. The sodium carbonate-treated exosomes were then resuspended in dI-ddH2O and assayed for HIV-1 suppression activity. An aliquot of each dI-ddH2O extracted exosome sample was lyophylized by speed-vaccum spin. Lyophilized samples were then resuspended in 3 μl of a solution of 0.3% Tricitric Acid/50% Acetylnitrile and then mixed with 3 μl of α-cyano-4-hydroxycinnamic acid. Aliquots of 1.5 μl were spotted on a stainless steel mass spec plate and dried at 40° C. The matrix-embedded samples were then analyzed by MALDI-TOF on a Voyager DE-PRO Mass Spectrometer (Applied Biosystems, US).

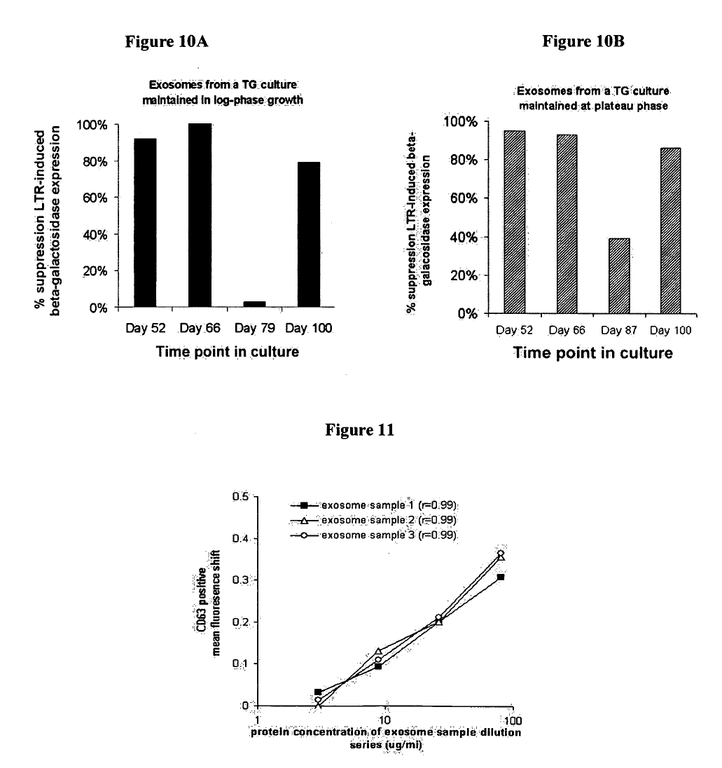

Results. Variability in exosome-mediated HIV-1 LTR promoter suppression activity. The investigation began by an analysis of possible fluctuations in exosome-mediated suppression of HIV-1 transcription. In the previous analysis of CD8+ cell culture fluids, it was observed that in two primary CD8+ T cell cultures examples of exosomes containing no LTR suppressive activity indicating that exosome-mediated HIV-1 suppression activity was not consistent (see FIGS. 9A-B). It was not known at the time whether the LTR suppression activity fluctuated with respect to its exosome localization in the TG cell line. If it did, exosome samples truly displaying divergent degrees of antiretroviral activity would provide an important starting point for dissecting the reasons underlying exosome localization of LTR suppression activity. Therefore, time-course analysis of the exosome-mediated antiretroviral activity was performed in two independent TG cultures that were at late stages of culture. Exosome purifications were performed at four independent time points for each culture and exosome samples were standardized by protein content. The HIV-1 LTR suppression activity of each purified sample was assayed in the acute LTR suppression assay utilizing the TZM-b1 cell line. Two instances were found over a time course from day 52 to day 100 where exosome-mediated LTR suppression activity fluctuated in both TG cultures (FIGS. 10A and B).

The analysis indicated that fluctuations of exosome-mediated LTR suppression did occur, however, a determination was sought to exclude the possibility that this variability was due to differences in exosome concentration in the samples standardized by protein concentration. To address this, an exosome titration assay was employed based on the quantitative immunomagnetic bead-capture method of Clayton et al., J Immunol Methods, 247(1-2):163-74 (2001). The quantitative detection of exosomes using anti-MHC Class II antibody-coated beads is based on the principal that under conditions of unsaturated bead capture of exosomes, flow cytometric measurement of exosome markers produces a fluoresence shift relative to isotype control that is directly proportional to concentration of exosomes during bead binding, Clayton et al., J Immunol Methods, 247(1-2): 163-74 (2001). The utility of this assay was demonstrated on exosomes prepared from three independent TG cell cultures. A 2-fold dilution series of each of the three independent samples was prepared from 80 ug/ml to 10 ug/ml. Using quantitative exosome capture assay, a striking linear correlation was found between exosome protein concentration and CD63 fluoresence shift (FIG. 11), demonstrating the utility of this assay. The concordance between the three independent exosome samples indicated a high degree of reproducibility of the quantitative exosome capture assay.

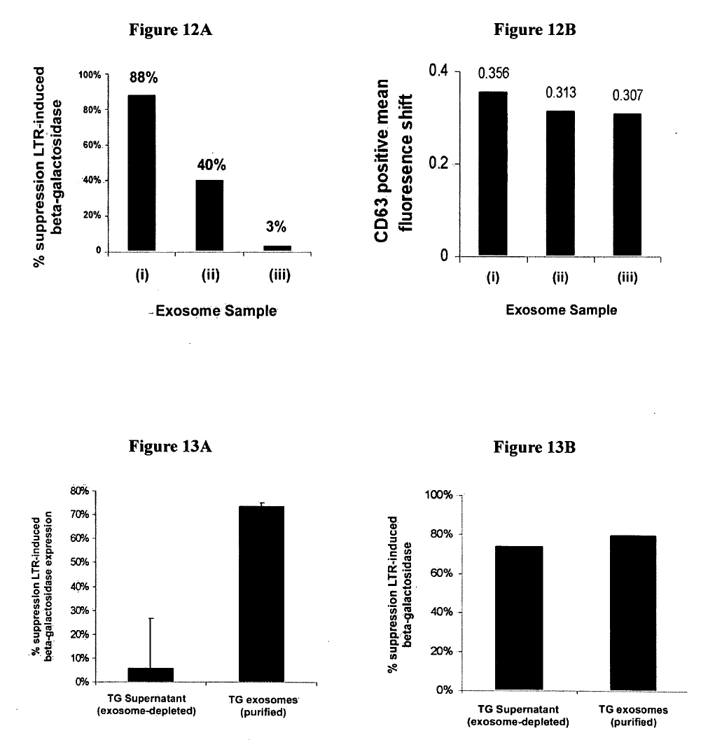

With an exosome quantitative assay on hand, three particular exosome samples were analyzed from the combined set displaying high, medium and low activities LTR suppression activities (FIG. 12A). Using the quantitative exosome assay, it was found that these three samples contained equivalent amounts of exosomes as indicated by similar CD63 dependent shifts (FIG. 12B). This demonstrated that the fluctuation of exosome-mediated HIV-1 LTR suppression activity was specifically due to the variable presence of a factor on the exosomes themselves and not to differences in exosome concentration or method of standardization.

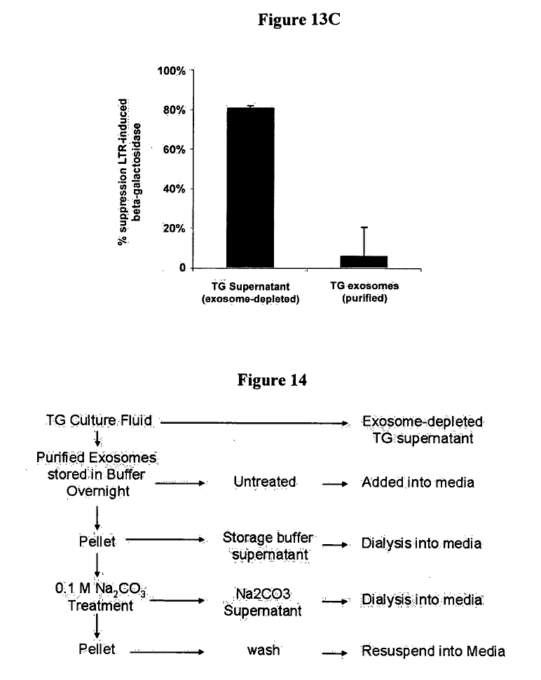

After determining the specific variability of a factor localizing to exosomes, the possible relationship was probed of variable TG exosome-mediated antiretroviral activity with concurrent activity in exosome-depleted culture supernatant. Upon analysis of several independent samples, instances were observed where LTR suppression activity was found exclusively in the exosomes (FIG. 13A), instances where LTR suppression activity was localized to both supernatant and exosomes (FIG. 13B), and instances where suppression activity was found only in the supernatant and not exosomes (FIG. 13C). The results indicate that fluctuations in exosome-mediated antiretroviral activity do occur and a pattern of inverse association between exosome-localized LTR suppression activity and the appearance of a soluble mediator in exosome-depleted culture supernatant could be found.

Nature of the LTR suppressing factor's localization to TG exosomes. The apparent fluctuation of the LTR suppressing activity between an exosome-localized and soluble form prompted an evaluation to more precisely define the nature of the LTR-suppressive activity's localization to exosomes. If the factor was indeed a cleavable precursor, the soluble form of the activity might still be localized as a loosely bound peripheral membrane protein on the exosomes. Furthermore, the extent of an integral membrane protein LTR suppressing activity present in exosomes should correlate inversely with the presence of a soluble mediator on the exosomes and in exosome-depleted culture supernatant. An analysis was performed on two exosome samples purified from two independent TG cultures in which one culture displayed considerable LTR suppression activity in exosome-depleted culture fluid and a second culture displaying no such activity from a soluble protein mediator in exosome-depleted culture fluid. The two exosome samples were subjected to a variety of salt treatments to quantify the extent of LTR suppression activity that was soluble and that which remained membrane-bound after treatment.

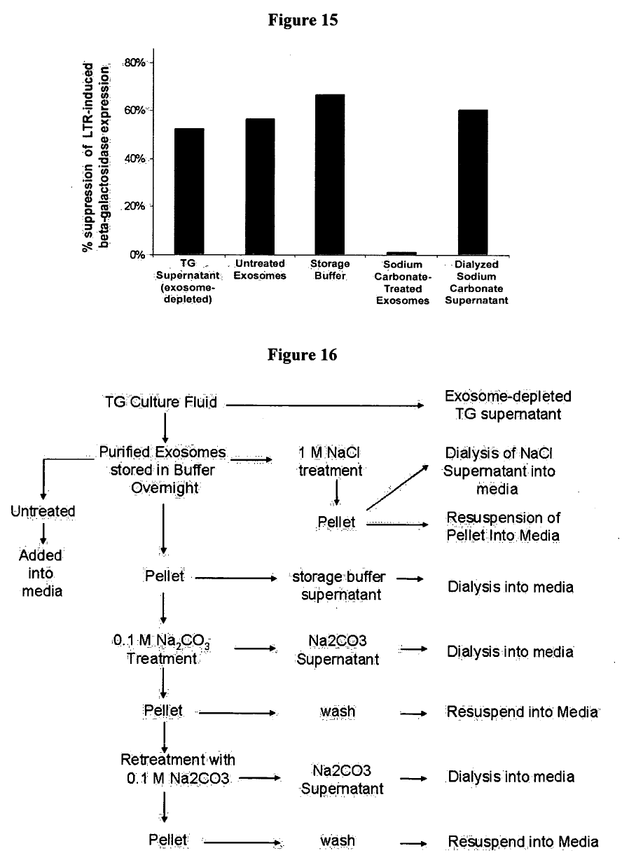

In the first exosome sample where significant LTR suppressing activity was found in exosome-depleted culture fluid, exosomes were subject to a series of soluble extractions as outlined in FIG. 14. After purification from cell culture fluid, exosomes were stored in HANKS buffer overnight with an aliquot of exosome-depleted culture supernatant saved for analysis. An untreated aliquot of the exosome suspension was also saved as a control before the remaining suspension was centrifuged to separate the exosomes and extract the storage buffer supernatant, which was saved for analysis. The exosome pellet was treated with 0.1 M sodium carbonate, pH 11.5 to remove all remaining peripheral proteins. After treatment, exosomes were pelleted and supernatant of the sodium carbonate extract and the exosome storage buffer supernatant were separately dialyzed into media. The sodium carbonate-treated exosome pellet was washed and resuspended into media. The LTR suppression activity was assayed in each of the fractions collected and found no activity in exosomes after sodium carbonate treatment (FIG. 15). The LTR suppression activity was only found in dialyzed sodium carbonate fractions and storage buffer supernatant in addition to its appearance in culture supernatant. In this particular exosome sample, the activity was found to be completely localized to exosomes as a loosely bound peripheral protein.

A similar analysis was performed on exosomes that were prepared from a second TG cell culture in which no LTR suppressing activity could be found in culture supernatants. The experimental schema for the second analysis is outlined in FIG. 16. This second treatment was a much more rigorous analysis to ensure that extraction of peripheral proteins was complete and exhaustive, therefore aliquots of exosomes were also subjected to 1M sodium chloride extraction and two serial sodium carbonate extractions were performed to ensure thorough removal of soluble proteins. After harvesting the various soluble extractions and treated exosome fractions, they were assayed for LTR suppression activity to determine if our model held for a cleavable factor held true.

It was found that LTR suppression activity could be eluted from this second exosome sample by sodium chloride treatment and sodium carbonate treatments in addition to its elution into the exosome storage buffer (FIG. 17A). However, it was surprisingly found that LTR suppression activity was also extracted into solution after two successive rounds of sodium carbonate treatment of the exosome samples (FIG. 17A). When assaying the suppression activity of resuspended post-treatment exosome pellet fractions, it was found that in contrast to the previous exosome samples, the second exosomes retained membrane-localized LTR suppression activity throughout all salt treatments, even after two successive rounds of sodium carbonate treatment (FIG. 17B). The results of this second analysis demonstrated a tight association of the peripheral membrane protein mediating the LTR suppression activity to the exosomes since successive treatments with sodium carbonate, a harsh alkali which thoroughly dissociates peripheral proteins from membrane association, was found to still elute a soluble LTR suppressive activity after the second treatment even after the first treatment should have removed all peripheral proteins from this exosome sample. Furthermore, a second sodium carbonate treatment did not completely remove the exosome-localized antiretroviral activity as evidenced by significant LTR suppression activity in exosomes after two successive treatments. Such a result is inconsistent with the soluble LTR suppressing protein associating to exosomes purely noncovalently.

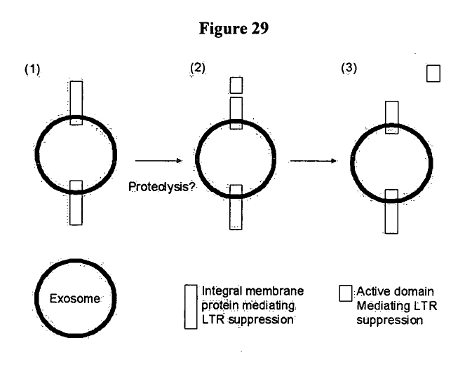

The results of the first analysis (FIG. 15) and the second analysis (FIGS. 17A-B) provide evidence for an antiretroviral factor that is localized to exosomes as both an integral and peripheral membrane protein. In the combined cases, the extent to which the LTR suppression activity is tightly associated to exosome membranes inversely correlates with the degree to which same antiretroviral activity appears in exosome-depleted culture fluids. Furthermore, the results in the second analysis argue against a purely non-covalent association of eluted LTR suppression activity. These results are consistent with a model of an integral membrane protein precursor containing an extracellular domain that can be cleaved into a separate protein fragment (FIG. 29).

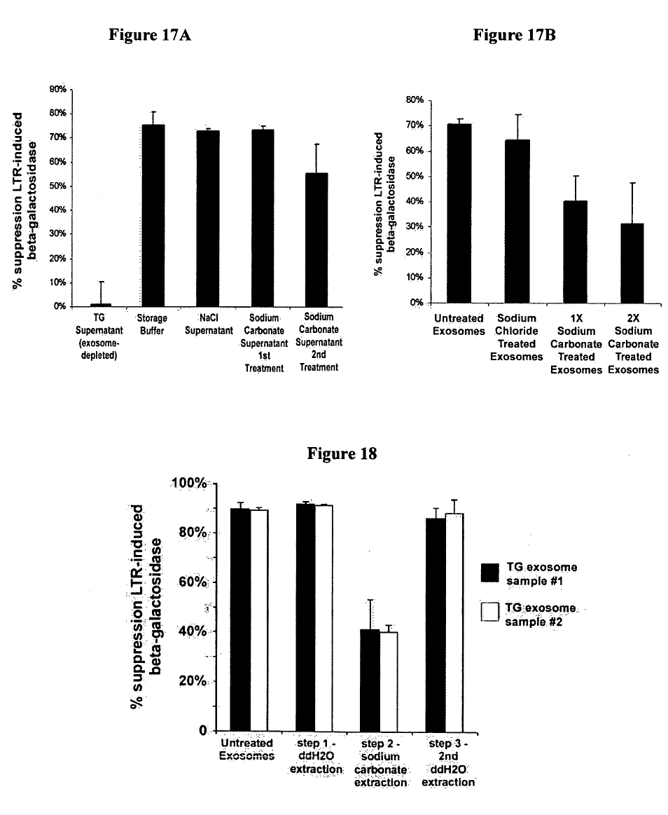

To confirm the validity of such a model for the exosome-bound LTR suppressing factor, exosomes were purified from two independent TG cell cultures, resuspended in dI-ddH2O (deionized double distilled water) and stored overnight at 4° C. The exosomes were pelleted and the supernatant was extracted. Exosome pellets were subjected to sodium carbonate treatment to remove any remaining peripheral proteins from the dI-ddH2O treated exosomes with the supernatant of the treatment dialyzed into buffer. The sodium carbonate treated exosomes were then resuspended in dI-ddH2O for a second extraction overnight at 4° C. After assaying LTR suppression activity of the various fractions, it was found that, in agreement with previous analysis, a high amount of antiretroviral activity, greater than what was eluted after sodium carbonate extraction of peripheral proteins from the exosomes, was extracted into solution (FIG. 18). This is further proof of the LTR suppressing factor existing as an integral membrane protein on the exosomes with its catalytical conversion into a soluble isoform.

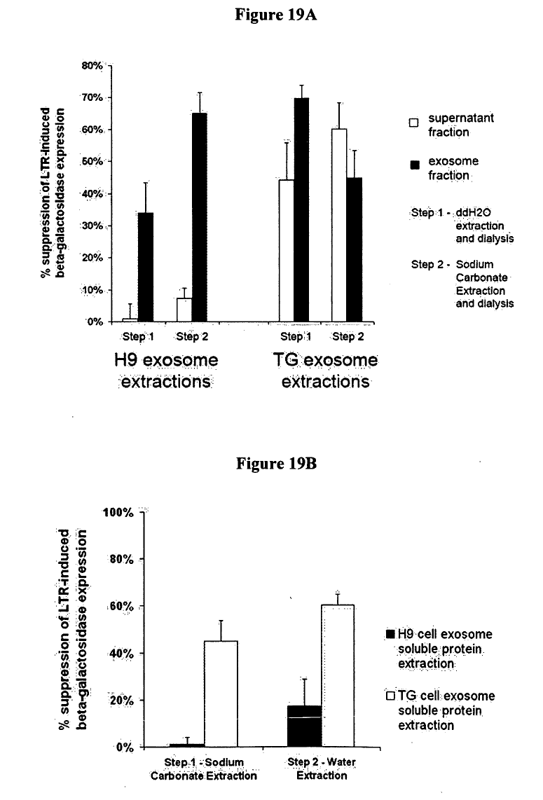

The analysis of the cleavable precursor model was extended to determine if the same might also be true for LTR suppression activity in exosomes from the H9 cell line since these CD4+ cell-secreted exosomes also displayed potent levels of the antiretroviral activity (FIG. 7A-B). Exosomes were purified from H9 and TG cell cultures and resuspended them in dI-ddH2O for extraction of soluble proteins. After overnight extraction at 4° C., exosomes were pelleted and supernatant was harvested. Pelleted H9 and TG Exosome were next subjected to sodium carbonate treatment upon which dialyzed supernatant and resuspended membrane pellets were prepared. After assaying the dI-ddH2O and sodium carbonate supernatant and pellet fractions, it was observed that extraction of a soluble form of the LTR suppressing activity, either by water or sodium carbonate extraction, was restricted only to exosomes prepared from the TG cell line, while both TG and H9 exosome membrane fractions displayed comparable LTR suppression activity after the successive extractions (FIG. 19A). In a second experiment on another set of H9 and TG exosome samples, the order of soluble extractions was reversed by first treating with sodium carbonate followed by extraction with dI-ddH2O. Results of this experiment further demonstrated that production of the solublized LTR suppression activity was largely restricted to TG exosomes (FIG. 19B). These results demonstrate that only TG exosomes contained significant catalytic activity to convert an integral membrane-bound form of the LTR suppressing activity into a soluble form. This proteolytic activity appears to be absent or greatly deficient in H9 exosomes since LTR suppression activity was retained in the H9 exosome membrane fraction after successive dI-ddH2O and sodium carbonate treatments (FIG. 19A).

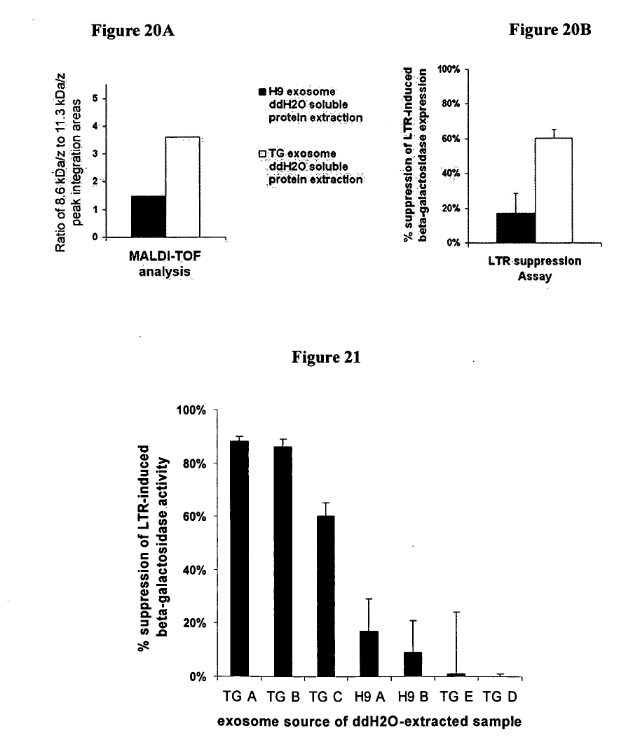

MALDI-TOF analysis of dI-ddH2O-eluted fractions from H9 and TG exosomes. The finding that the soluble form of the LTR suppressing activity was largely restricted to TG exosomes made H9 exosomes an ideal negative control for analysis of dI-ddH2O extracted samples by differential proteomic analysis. Exploitation of the proteomic analysis technique of matrix assisted laser desorption ionization-time of flight (MALDI-TOF) was sought to determine if differences in LTR suppression activity could be correlated to differential MALDI-TOF analyte peaks produced from proteins in the dI-ddH2O extracted samples. The dI-ddH2O extracted TG and H9 samples were analyzed described in FIG. 22.B by MALDI-TOF using an Applied Biosystems Voyager Mass Spetrometer. In the resulting spectra, a mass/charge (m/z) range was analyzed between m/z 3.5 kDa and m/z 14.0 kDa in order to identify differential and common peaks between the TG and H9 samples. Observed was a common triplet of peaks in the two samples of m/z 11.3 kDa, m/z 11.7 kDa, and m/z 12.2 kDa in both the TG and H9 solublized samples. The m/z 11.3 kDa peak was chosen to serve as an internal control in attempting to identify possible differential peaks between the TG and H9 samples. Since the samples analyzed were standardized for volume and were extracted from their exosome sources at equivalent exosome protein concentrations, differentially displayed analyte peaks relative to an internal control should reflect the relative levels of the protein giving rise to a particular peak. Of interest in the analysis were MALDI-TOF peaks that were at higher levels in the TG sample than in the H9 relative to the 11.3 peak was chosen as an internal control for both spectra. One such peak at m/z 8.6 kDa appeared to be higher in the TG spectra than for the H9 spectra. The ratio of the peak integration values of m/z 8.6 kDa to m/z 11.3 kDa analytes (FIG. 20A) corresponded strikingly with the differential LTR suppression activity observed between the TG and H9 dI-ddH2O extracted samples (FIG. 20B).

The MALDI-TOF analysis was expanded to a larger panel consisting of dI-ddH2O extractions from five TG and two H9 exosome samples. The seven dI-ddH2O extracted samples displayed a divergent range of LTR suppression activity (FIG. 21). MALDI-TOF analysis was performed on the seven samples. It was observed the characteristic triplet peaks of m/z 11.3 kDa, m/z 11.7 kDa, and m/z 12.2 kDa in all seven samples analyzed, validating their use as internal controls. In addition to analysis of the m/z 8.6 kDa peak, also identified were m/z 5.0 kDa, m/z 5.4 kDa, and m/z 6.2 kDa peaks that appeared to correlate with HIV suppressing sample activity.

Since MALDI-TOF analyte peaks correspond to proteins contained in the original exosome extracts, relative peak integrations standardized by the m/z 11.3 kDa internal control as well as the original exosome protein concentration during dI-ddH2O extraction of the fractions, describe the relative concentration of a protein giving rise to a specific mass/charge peak. Calculation of relative protein concentrations corresponding to m/z 5.0 kDa, m/z 5.4 kDa, m/z 6.2 kDa and m/z 8.6 kDa peaks (FIGS. 22.A-D) were striking in their correspondence to LTR suppression activity (FIG. 20B) for the panel of samples analyzed. These data do not necessarily implicate any one of these peaks to be the actual protein mediating LTR suppression. They are however clear markers of a common proteolytic action that correlate with release of the soluble protein mediating LTR suppression. Interestingly, the relative relationship of m/z 5.0 kDa, m/z 5.4 kDa, m/z 6.2 kDa and m/z 8.6 kDa peaks quantitatively correspond to an average ratio of 3:1:1:2 in the four samples expressing significant LTR suppression activity (Table 1). This would identify the four peaks as a functional set, since the proportions are roughly conserved in the four samples displaying significant activity, compared to other peaks, such as the m/z 11.3 kDa, which appears invariant to LTR suppression activity or the m/z 5.0 kDa, m/z 5.4 kDa, m/z 6.2 kDa and m/z 8.6 kDa quadruplet. The presence of such a functional set and the correspondence of these peaks with LTR suppressive defines a marker for a specific proteolytic activity that cleaves the protein(s) giving rise to these four MALDI-TOF peaks and the solublized LTR suppressive activity.

TABLE 1

| | m/z 5.0 kDa | m/z 5.4 kDa | m/z 6.2 kDa | m/z 8.6 kDa |
|---|---|---|---|---|
| | (relative ratio) | (relative ratio) | (relative ratio) | (relative ratio) |
| Sample I | | | | |
| TG A | 3.240964 | 1 | 1.018072 | 2.230924 |
| TG B | 2.702479 | 1 | 1.024793 | 2.427686 |
| TB C | 3.444444 | 1 | 1.160494 | 2.345679 |
| H9 A | 2.428954 | 1 | 0.780161 | 1.123324 |
| Average | 2.954211 | 1 | 0.99588 | 2.031903 |
| Sample II | | | | |
| TG A | 3.183432 | 0.982249 | 1 | 2.191321 |
| TG B | 2.637097 | 0.975806 | 1 | 2.368952 |
| TB C | 2.968085 | 0.861702 | 1 | 2.021277 |
| H9 A | 3.113402 | 1.281787 | 1 | 1.439863 |
| Average | 2.975504 | 1.025386 | 1 | 2.005353 |

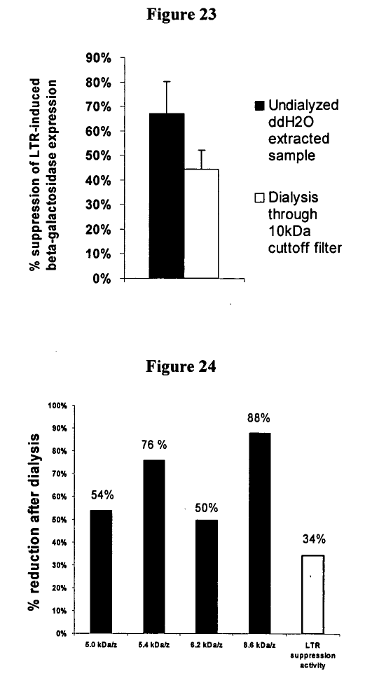

In order to determine if any of these peaks might be directly related to the LTR suppressing activity, a dialysis was performed to determine if retention of the identified peaks coincided with retention of LTR suppression activity. A fresh sample of TG exosomes was purified and subjected to sodium carbonate treatment to remove all peripheral proteins followed by extraction with deionized double distilled water (dI ddH2O). The dI ddH2O-extracted fraction was then dialyzed against deionized water for 4 hours using a 10 kDa cutoff Pierce dialysis membrane cassette. An aliquot of undialyzed dI-ddH2O fraction was saved as a control. The fractions were assayed for LTR suppression activity in addition to analysis by MALDI-TOF. It was found that dialysis through a 10 kDa cutoff membrane lead to a moderate loss of LTR suppression activity (FIG. 23), indicating that the soluble protein responsible for the antiretroviral activity is only partially retained by the 10 kDa cutoff membrane cassette.

In MALDI-TOF analysis of dialyzed and undialyzed samples, an apparent decrease in the characteristic m/z 5.0 kDa, m/z 5.4 kDa, m/z 6.2 kDa and m/z 8.6 kDa marker peaks relative to the m/z 11.3 kDa control peak was noted in the 10 kDa cutoff dialyzed samples compared to undialyzed control (FIG. 23). A general reduction in these analyte signals in dialyzed samples compared to undialyzed control corresponded with the loss of LTR suppression activity (FIG. 23). An additional analyte signal of m/z 2.5 kDa was also detected which also appeared to roughly correlate with anti-HIV activity of the soluble fractions.

Therefore, this example demonstrates the mechanistic relationship between the exosome-mediated LTR suppressing activity and its appearance as a soluble protein. Clear evidence of a molecular relationship between the two demonstrates that a soluble LTR suppressing factor is directly produced from a membrane bound precursor also exhibiting the same activity.

### EXAMPLE 3

This example demonstrates the pH and heat stability of the compound according to one embodiment of the invention.

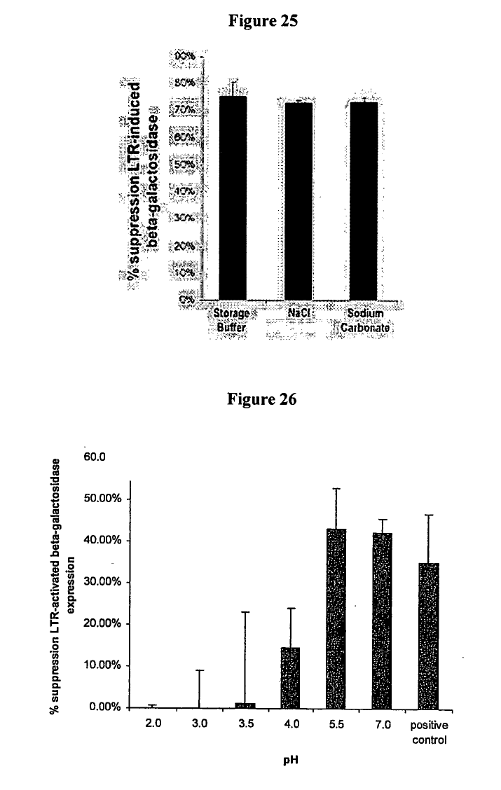

An exosome purification was performed from a TG cell culture according to example 2. Three aliquots of exosomes were pelleted by centrifugation and resuspended either in storage buffer (pH 7) for 30 min, in 1 M NaCl solution (pH 7) for 30 min, or in 0.1 M sodium carbonate (pH 11.5) for 30 min, to extract the soluble form of the antiretroviral protein from the exosome membrane. After the extractions, all three samples were dialyzed by centrifugal filtration into storage buffer, adjusted to equivalent volume, and assayed for HIV-1 promoter suppression activity. Equivalent suppression activity was recorded for all three samples indicating the complete stability of the antiretroviral protein at pH 11.5 (FIG. 25)

In another study, aliquots of the water extraction were made. Various pH solutions of 0.1% trifluoroacetic acid (TFA) were prepared as set forth in Table 2. A set of aliquots containing the soluble antiretroviral protein were dialyzed into one of the low pH buffers with a control aliquot kept on ice. After a 30 min incubation at room temperature, pH-treated aliquots were dialyzed into neutral pH buffer (HANKS balanced salt solution) and assayed for HIV-1 LTR promoter suppression activity.

Full HIV suppression activity was retained for pH 7.0 and 5.5. At pH 4.0 treatment, HIV suppression activity diminished by 60-68% compared to pH 7.0 and the positive control kept on ice. HIV suppression was lost at a pH below 4.0 (see FIG. 26).

TABLE 2

| pH | TFA | HEPES buffer |
|---|---|---|
| 2.0 | 13 mM | 0 mM |
| 3.0 | 13 mM | 10 mM |
| 3.5 | 13 mM | 20 mM |
| 4.0 | 13 mM | 30 mM |
| 5.5 | 13 mM | 40 mM |
| 7.0 | 13 mM | 50 mM |

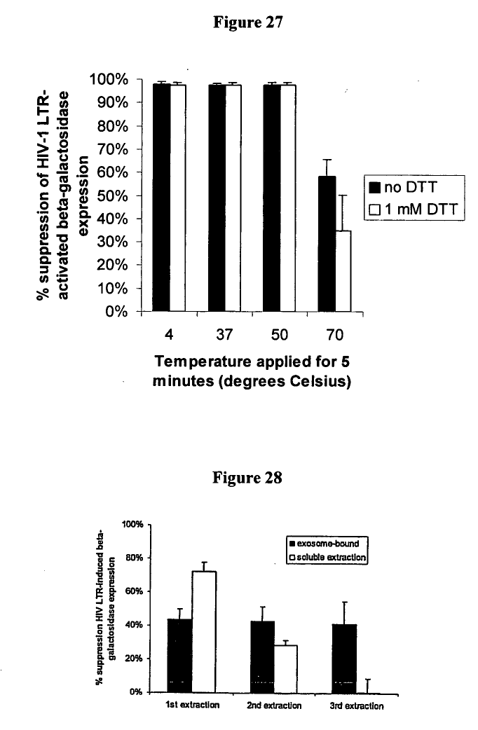

A set of 30 aliquots of sample containing a high amount of anti-HIV activity was subjected to one of the following treatments: 4° C. for 5 min (positive control), 37° C. for 5 min, 50° C. for 5 min, or 70° C. for 5 min using a Perkin Elmer thermocycler either in the presence or absence of 1 mM DDT. For the 4° C. positive control, 98% suppression of the HIV-1 promoter was recorded. This activity was completely maintained after applying a 5 min temperature treatment of either 37° C. or 50° C. With a temperature treatment of 70° C., the HIV-1 promoter suppression activity was reduced to 58%±7% in the absence of DDT and 35%±16% in the presence of DDT (FIG. 27).

### EXAMPLE 4

This example demonstrates that the antiretroviral protein may be extracted and re-extracted.

In one sample, three sequential water extractions were performed on a TG exosome prepared according to Example 2. The HIV transcription suppression activity of both soluble and exosome fraction were determined for each of the three sequential extractions. The results are shown in FIG. 28.

### EXAMPLE 5

This example demonstrates that the inventive antiretroviral polypeptide retains its activity following lyophilization and reconstitution.

An extract of the soluble antiretroviral protein was made as follows: Purified exosomes were first subjected to 0.1 M Sodium Carbonate treatment for removal of peripheral proteins from the exosomes. Exosomes were then pelleted, washed, and resuspended in de-ionized double distilled water at a protein concentration of 1 mg/ml. The water resuspended exosomes were incubated at 4° C. for 24 hours. After incubation, the exosomes were pelleted by centrifugation and the aqueous supernatant containing the antiretroviral polypeptide was extracted.

A 30 μl aliquot of the extract was stored at 4° C. as a positive control. A second 30 μl aliquot was placed in a Speed Vac rotor and maintained under vacuum conditions until the sample was dried off and all liquid was removed from the sample. The lyophilized protein was resuspended in 30 μl of de-ionized double distilled water.

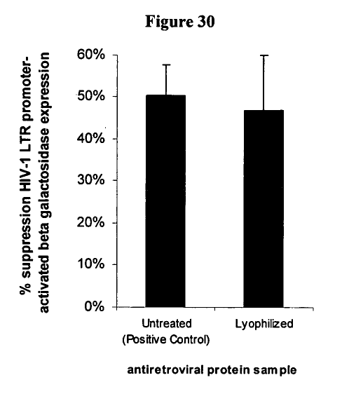

Both lyophilized sample and positive control were assayed for HIV-1 LTR promoter suppression activity in TZM-b1 cells. It was observed that the antiretroviral activity of the protein was preserved following lyophilization. These results are shown in FIG. 30.

### EXAMPLE 6

This example demonstrates that the inventive antiretroviral polypeptide is inactivated by trypsin and chymotrypsin.

An extract of the soluble antiretroviral protein was made as described in Example 5. From this extract, an aliquot of 30 μl containing the antiretrovial protein was incubated at 37° C. for 18 hours as a positive control. A second aliquot of 30 μl containing the antiretroviral protein was incubated at 37° C. for 18 hours with trypsin at a concentration of 5 μg/ml trypsin enzyme. A third aliquot of 30 μl containing the antiretroviral protein was incubated at 37° C. for 18 hours with chymotrypsin at a concentration of 5 μg/ml chymotrypsin enzyme.

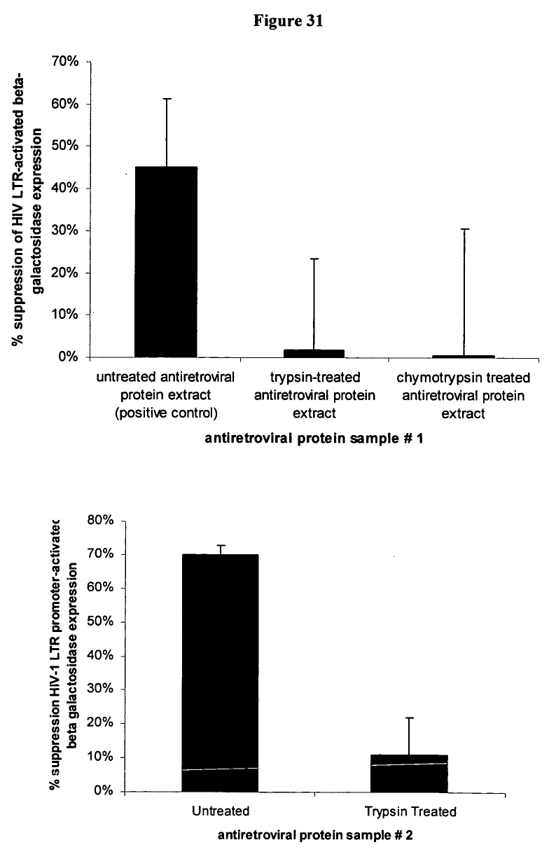

After the 18 hour incubation of positive control, trypsin-treated, and chymotrypsin-treated samples, aliquots of each sample were directly assayed for HIV-1 LTR promoter suppression activity in TZM-b1 cells. It was observed that the trypsin-treated, and chymotrypsin-treated samples did not suppress LTR promoter activity, whereas the positive control did. These results are shown in FIG. 31.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or substantially purified antiretroviral polypeptide that suppresses the activity of a retroviral long terminal repeat promoter (LTR) promoter and which possesses at least one of the following characteristics:
a. The antiretroviral polypeptide is less than about 13 kDa in size;
b. The antiretroviral polypeptide is pH stable between about pH 4 through about pH 11.5; and
c. The antiretroviral polypeptide is sensitive to trypsin.

2. The antiretroviral polypeptide of claim 1, which possesses two or more of the listed characteristics.

3. The antiretroviral polypeptide of claim 1, which possesses each of the listed characteristics.

4. The antiretroviral polypeptide of claim 1, wherein the polypeptide exhibits a mass/charge (m/z) value of about m/z 2.5±0.1 kDa as determined by MALDI-TOF mass spectrometry.

5. The antiretroviral polypeptide of claim 1, wherein the polypeptide exhibits a m/z value of about 5.0±0.1 kDa as determined by MALDI-TOF mass spectrometry.

6. The antiretroviral polypeptide of claim 1, wherein the polypeptide exhibits a m/z value of about 5.4±0.1 kDa as determined by MALDI-TOF mass spectrometry.

7. The antiretroviral polypeptide of claim 1, wherein the polypeptide exhibits a m/z value of about 6.2±0.1 kDa as determined by MALDI-TOF mass spectrometry.

8. The antiretroviral polypeptide of claim 1, wherein the polypeptide exhibits a m/z value of about 8.6±0.1 kDa as determined by MALDI-TOF mass spectrometry.

9. The antiretroviral polypeptide of claim 1, which is water soluble.

10. The antiretroviral polypeptide of claim 1, which is heat stable.

11. The antiretroviral polypeptide of claim 1, which is retained by a 5 kDa microfilter cassette.

12. The antiretroviral polypeptide of claim 1, which is derived from CD8+ T lymphocytes, CD4+ T lymphocytes, B lymphocytes, or transformed cells thereof.

13. The antiretroviral polypeptide of claim 1, which is derived from a cell membrane, a cell surface, an endosomal compartment, a microvesicle, an exosome, or a combination of thereof.

14. The antiretroviral polypeptide of claim 1, which retains anti-retroviral activity after lyophilization.

15. The antiretroviral polypeptide of claim 1, which is sensitive to chymotrypsin.

16. The antiretroviral polypeptide of claim 1, wherein the polypeptide suppresses retroviral gene expression.

17. The antiretroviral polypeptide of any of claims 1-9, wherein the polypeptide suppresses replication of HIV, SIV, or HTLV.

18. The antiretroviral polypeptide of claim 17, wherein the polypeptide suppresses replication of HIV.

19. The antiretroviral polypeptide of claim 18, wherein the polypeptide suppresses replication of HIV-1.

20. The antiretroviral polypeptide of claim 18, wherein the polypeptide suppresses replication of HIV-2.

21. A composition comprising the antiretroviral polypeptide of any of claims 1-9 in the absence of CD8+ T lymphocytes, CD4+ T lymphocytes, or B lymphocytes.

22. A composition comprising the antiretroviral polypeptide of any of claims 1-9 at least 99% purified from other proteinaceous material.

23. A composition consisting essentially of the antiretroviral polypeptide of any of claims 1-9, water, and optionally a buffer.

24. A composition comprising the antiretroviral polypeptide of any of claims 1-9 in lyophilized form, optionally comprising a lyoprotectant.

25. A method of inhibiting retroviral replication, wherein the method comprises administering an antiretroviral polypeptide according to any of claims 1-9 to a cell infected with a retrovirus in an amount sufficient to inhibit replication of the retrovirus within the cell.

26. The method of claim 25, wherein the antiretroviral polypeptide is administered in vitro.

27. The method of claim 25, wherein the antiretroviral polypeptide is administered in vivo.

28. The method of claim 25, wherein the antiretroviral polypeptide is administered to a human.

29. The method of claim 28, wherein the retrovirus is HIV.

30. A pharmaceutical composition comprising the antiretroviral polypeptide according to any of claims 1-9 and a pharmaceutically-acceptable excipient, diluent or carrier.

31. A method of treating a subject infected with a retrovirus, the method comprising administering a therapeutically effective amount of a composition comprising the pharmaceutical composition of claim 30 in an amount sufficient to treat the retroviral infection within the subject.

32. The method of claim 31, wherein the subject is human and the retrovirus is HIV.

33. The method of claim 32, wherein the HIV is HIV-1

34. The method of claim 32, wherein the HIV is HIV-2.

35. A method of diagnosing an infection with a retrovirus, the method comprising detecting the presence of the antiretroviral polypeptide of any of claims 1-9 in a sample derived from a subject, and wherein the presence of the antiretroviral polypeptide is correlated with an infection with a retrovirus within the subject.

36. A method for extracting peptides localized to cell exosomes, which method comprises (a) purifying exosomes from cells; (b) adding storage buffer to the purified exosomes; (c) treating the exosomes with a high molarity salt solution; (d) pelleting the exosomes by centrifugation; (e) extracting the supernatant from the treated exosomes, wherein the supernatant comprises soluble peptides; and (f) optionally dialyzing the supernatant into an aqueous media to collect the extracted peptides.

37. The method of claim 36, wherein the salt solution is about 1 M NaCl.

38. A method for extracting peptides localized to cell exosomes, which method comprises (a) purifying exosomes from cells; (b) adding storage buffer to the purified exosomes; (c) pelleting the exosomes by centrifugation; (d) treating the centrifuged pellet of exosomes with a high pH composition; (e) extracting the supernatant from the treated exosomes, wherein the supernatant comprises soluble peptides; and (f) optionally dialyzing the supernatant into an aqueous media to collect the extracted peptides.

39. The method of claim 38, wherein the high pH composition has a pH of at least about 11.

40. The method of any of claims 36-39, wherein method steps (b-f) are repeated.

41. The method of any of claims 36-39, wherein the cell exosomes are derived from cells selected from the group consisting of CD8+ T lymphocytes, CD4+ T lymphocytes, B lymphocytes, and transformed cells thereof.
